(19) United States
(12) Patent Application Publication
(10) Pub. No.: US 2012/0309052 A1
(43) Pub. Date: Dec. 6, 2012

(54) INDUCIBLE BACULOVIRUS SYSTEM IN INSECT CELLS

(76) Inventors: Josef Daniel Fitzgerald, Geneva (CH); Bernd Walzel, Zurich (CH)

(21) Appl. No.: 13/575,183

(22) PCT Filed: Jan. 25, 2011
(86) PCT No.: PCT/EP2011/050996

§ 371 Date: Jul. 25, 2012

Related U.S. Application Data
(60) Provisional application No. 61/297,904, filed on Jan. 25, 2010.

(51) Int. Cl. C12N 15/85; C12P 21/00; C12N 5/10

(52) U.S. Cl. 435/69.1; 435/320.1; 435/348

(57) ABSTRACT

The present invention describes a novel baculovirus protein expression system in insect cells, where the expression of recombinant protein(s) is inducible. By the inducible expression systems of the invention, expression of the recombinant protein of interest can be repressed during virus amplification, and thereafter activated in the presence or absence, respectively, of an inducing molecule or by a change in environmental conditions. The novel expression system does not produce significant levels of recombinant protein by baculovirus-infected cells during virus amplification prior to induction, thereby reducing selection pressure on the expression cassette for the recombinant protein. When induction is initiated, an increased yield of recombinant protein is thus produced relative to presently available, non-inducible baculovirus expression methods. Further, it is demonstrated herein that the yield of protein expression derived from the described methodology further increases relative to non-inducible systems as the scale of protein expression increases.

### FIELD OF THE INVENTION

The invention relates to an inducible baculovirus protein expression system for improved recombinant protein production, wherein expression of the recombinant protein can be repressed during virus amplification and induced during the recombinant protein production phase.

### BACKGROUND OF THE INVENTION

Recombinant Protein Expression: A vast number of expression systems are used to produce recombinant proteins, ranging from cell free systems to cell based systems. Presently, due to technical limitations associated with cell free expression systems, cell based systems are more commonly used for recombinant protein expression. Cell based expression systems include those utilizing bacteria, yeast, insect cells or mammalian cells as hosts. The majority of these systems utilize inducible expression. For example, in a recent international collaboration to produce and purify over 10,000 recombinant proteins for use in structural biology, well over 90% of these proteins were produced using some form of inducible expression system (Nature Methods 5, 135-146).

Probably the best known inducible recombinant protein expression system is the pET expression system. Here, in E. coli, a plasmid DNA carrying an expression cassette coding for the recombinant protein (gene) of interest is maintained in the E. coli host cell through a combination of an antibiotic present in the growth media, and a second expression cassette (gene) present on the expression plasmid which affords the cell carrying the plasmid resistance to the antibiotic present in the media (J. Mol. Biol. 189, 113-130). Transcription of the recombinant protein encoded (carried) by the plasmid is sterically inhibited by the presence of the lac repressor protein bound to its consensus DNA binding site on, or adjacent to, the lac promoter. Upon addition of an inducer molecule to the culture media, usually the lactose analogue IPTG, the inducer is taken up by the E. coli, binds to the lac repressor protein, and causes the lac repressor to dissociate from its DNA binding site in the recombinant protein expression cassette. Transcription and translation of the recombinant protein then rapidly follow. Using this method, E. coli culture volume can be scaled up to the desired size over a period of hours or days without significant production of the recombinant protein of interest. This ability to scale-up culture size without recombinant protein production allows large scale production of recombinant proteins that are toxic to E. coli and would otherwise inhibit scale-up. In addition, since recombinant proteins are by definition not essential for the survival of E. coli, blocking their expression during scale-up of E. coli culture size reduces selective pressure for cells to eliminate the non-essential recombinant protein expression cassette, and/or to accumulate mutations within the cassette that eliminate or reduce the expression of the cassette. The pET system has proven itself to be highly amenable to scale-up, as protein yield per culture volume does not greatly drop off during scale-up from milliliters to hundreds of liter scales.

Another commonly used recombinant protein expression system is the baculovirus insect cell expression system. Baculoviruses have very species-specific tropisms among invertebrate cells and are not known to replicate in mammalian or other vertebrate animal cells. For this reason of safety, and for a number of other reasons, the baculovirus expression system has become one of the most widely used systems for production of recombinant proteins (Nature Biotechnology 23, 567-575). A number of technological improvements have eliminated the original tedious procedures required to create and culture recombinant baculoviruses. Baculovirus expression holds a number of significant advantages over other eukaryotic expression systems. Among these are high recombinant protein yield, lower cost relative to mammalian expression systems, and the relatively low protease activity present in insect cells in comparison to yeast expression systems. In addition, for many purposes, the baculovirus system produces authentic post-translational modifications (see Nature Biotechnology 23, 567-575). The baculovirus Autographa californica multicapsid nuclear polyhedrosis virus (AcMNPV) is by far the most common vehicle in this system.

The baculoviruses are a family of large, rod-shaped, enveloped viruses that contain circular double-stranded DNA genomes ranging from 80-180 kilo base pairs (kbp). Baculovirus infection of host cells can be divided to three distinct phases: “early” (0-6 h post-infection (p.i.)), “late” (6-24 h p.i.) and “very late” (18-24 to 72 h p.i.). The baculovirus “very late” promoters display extremely high rates of transcription relative to host cell promoters and early or late baculovirus promoters. Therefore, the basic idea behind prior-art baculovirus expression of recombinant proteins in insect cells is that the DNA sequence coding for a recombinant protein of interest is shuttled into the baculovirus genome under the control of such a “very late” promoter, which results in high levels of expression of the recombinant protein during (very late stages of) infection of the cultured (host) insect cell.

Modern baculovirus expression systems allow for recombinant genes to be shuttled into the baculovirus genome through recombination of baculovirus DNA and recombinant gene-containing plasmids in insect cells, in bacteria, or in vitro. Using any of these methods, sufficient recombinant protein-encoding viruses are typically generated to infect insect cell culture volumes in the order of a few milliliters. However, for many applications, tens, hundreds, or even thousands of liters of insect cell culture are required to generate the desired levels of recombinant protein. One example for a large-scale recombinant protein production using a baculovirus expression system is the production of influenza vaccines (Protein Sciences Corp. (USA), www.proteinsciences.com). Scale-up for large scale baculovirus expression requires an exponential increase in the number of recombinant viruses from the starting point of small numbers of recombinant viruses, such as from a small pool on the order of millions of virus particles. Scale up is then typically carried out by allowing the recombinant viruses to replicate in sequentially larger insect cell numbers and/or culture volumes. Significantly, baculovirus protein production yield at small scale (e.g. milliliters) is typically observed to be up to several fold higher than at tens or hundreds of liter scale. Specifically, yield of protein produced per volume cell culture decreases stepwise per virus generation even when very strict precautions are followed (see O'Reilly et al. Oxford University Press, New York, 1994). It has even been argued that one potential contribution to this problem is simply an intrinsic property of the baculovirus system itself, since defective interfering (DI) viruses exponentially build up as baculoviruses undergo the multiple cycles of replication required to scale-up protein production (Biotech. Letters 13. 483-488; Virology 283, 132-138; Journal of General Virology, 84, 2669-2678). DI viruses are mutant viruses, often carrying large (up to 40%) deletions of their DNA (Virology 283, 132-138). Since some fraction of DI viruses are expected to contain deletions of the recombinant protein expression cassette, an accumulation of DI viruses during virus amplification correlates with a decrease in recombinant expression yield. The accumulation of DI viruses can be reduced if the experimenter carefully controls the the relative ratio of viruses to host cells during the scale up process.

In prior art baculovirus expression systems, the recombinant protein of interest is (initially) both heavily transcribed and translated by insect cells infected with baculovirus during virus amplification, and a second phenomenon, independent of DI virus accumulation, is the decrease of recombinant protein yield which accompanies baculovirus expression scale up.

Since the recombinant proteins produced by insect cell expression systems, including that driven by baculovirus very late promoters, are both 1) transcribed and translated at very high levels and therefore require a significant fraction of the cell's total metabolic capacity and 2) are not essential for replication of the virus, this results in a high selection pressure for production of viruses which do not carry their non-essential recombinant protein expression cassette or favouring mutant viruses which do not transcribe/translate the recombinant protein. It is expected that scaling up baculovirus expression to larger and larger culture volumes will rapidly select for, and/or result in a progressive accumulation of, viruses that do not carry the recombinant protein (DNA) expression cassette (Journal of General Virology, 84, 2669-2678; Nature Methods 3, 1021-1032), and/or of viruses that carry mutations in or around the expression cassette that do not (as efficiently) transcribe/translate the recombinant protein. This is not too surprising given the well documented phenomenon of plasmid loss experiments, which demonstrate that non-essential DNA is very rapidly removed from cells in the absence of selection pressure which maintains them (Plasmid 36, 161-167). In the case of specific elimination and/or mutation of recombinant protein DNA expression cassettes from baculoviruses during scale up, DNA recombination can be involved and therefore particular DNA sequences could potentially be hotspots for such recombination/mutation. Elimination of hyperrecombinic DNA sequences, particularly those of non-baculovirus origin such as the bacterial sequences contained in modern genetically engineered baculoviruses and recombinant protein transfer vectors, might thereby reduce the rate at which recombinant protein expression cassettes are eliminated and/or mutated from recombinant baculoviruses. Likewise, elimination of highly mutable DNA sequences anywhere within or adjacent to the recombinant DNA expression cassette is expected to slow the rate at which non-expressing mutant baculoviruses are generated during scale up.

The control of baculovirus very late promoter transcription has been intensively studied but remains not fully understood. The protein “vlf-1” appears to be the major transcriptional activator for very late promoters and acts by binding to defined DNA sequences within very late promoters and directly stimulating transcription (J. Virology 73, 3404-3409; J. Virology 79, 1958-60), in concert with a virally-encoded RNA polymerase (Virology 216, 12-19). Besides vlf-1, a number of other baculovirus-encoded factors are also known to be involved in regulating very late promoter transcription and include FP25 (Virology 226, 34-46), ie-1 (Virology 209. 90-98) Lef-2 (Virology 251, 108-122), Lef-4 (J Virol. 80, 4168-4173), PK-1 (Virus Res.135, 197-201) and Ac43 (Virology 392, 230-237). In contrast to vlf-1, none of these other factors have been shown to directly affect transcription levels of genes controlled by very late promoters, and may function through indirect mechanisms, e.g. by stimulating replication of baculoviral DNA. Finally, an enhancer-like DNA sequence has been identified upstream of the polh very late promoter which stimulates its transcription (J. Biol. Chem. 277, 5256-5264; J. General Virology 82, 2811-2819). Although not typically utilized for recombinant protein expression with baculovirus, late promoters or late/very late hybrid promoters have been shown to provide superior yield over very late promoters for particular recombinant proteins, such as membrane proteins. Factors that regulate baculovirus late promoters include Lef-1, Lef-2, Lef-3, Lef-4, Lef-5, Lef-6, Lef-7, Lef-8, Lef-9, Lef-10, Lef-11, Lef-12, IE-1, IE-2, Ac69, Ac38, Ac36, p47, p143, p35, DNAPOL, HCF-1. Further information on such factors can be found in J. Virology, 27: 10197-10206. Some factors (such as Lef-2 and Lef-4) regulate late and also regulate very late promoters of baculovirus. Baculovirus late and very late (or hybrid late/very late) promoters are distinguished from insect cell host promoters (or mammalian cell host promoters) in that they require factors encoded by the baculovirus genome.

There are a number of commercial systems available for expressing recombinant proteins using baculovirus, including flashBAC™ (Oxford Expression Technologies EP 1 144 666), BackPack™ (BD Biosciences Clontech), BacVector® 1000/2000/3000 (Novagen®). BAC-TO-BAC® (Invitrogen™ U.S. Pat. No. 5,348,886), and BaculoDirect™ (Invitrogen™m). All of these systems are based on the principle of expressing recombinant proteins by placing them under the control of the very late baculovirus promoters polh or p10. None of these systems allow for inducible control of protein expression, particularly, for repression of recombinant protein expression during virus amplification.

One baculovirus-based technology which does allow inducible control of protein production is the “BacMam” technology which uses baculoviruses as vehicles to deliver and inducibly express recombinant proteins in mammalian cells (Nature Biotechnol. 23: 567-575). This system does not allow inducible expression of recombinant proteins in insect cells, and does not use the heavily transcribed baculovirus very late promoters for recombinant protein expression; rather it uses mammalian promoters, because baculovirus very late promoters do not fire in mammalian cells. Corresponding approaches that use baculovirus technology to deliver an expression system into mammalian cells, for inducible expression of a recombinant protein in mammalian cells, have also been disclosed by McCormick et al (J Gen Virol, 2002; 83: 383-394 and J Gen Virol, 2004; 85: 429-439).

Aslanidi et at (PNAS, 2009; 106: 5059-5064) describe an inducible system for production of virus vectors in insect cells. Such system uses baculovirus as a gene transfer vector to, upon infection of insect cells by baculovirus, provide the baculovirus-encoded transcription factors that are required to carry out recombinant protein expression.

Wu et al., (Journal of Biotechnology, 80; 75-83) created an effective, plasmid based tetracycline regulatory expression system (TRES) for use in insect cells. However, high background activation of a minimal human cytomegalovirus immediate-early (CMVm) promoter by the viral polyhedrin upstream (pu) sequence precluded their parallel development, as reported in this publication, of a similarly effective inducible baculovirus expression system for insect cells. In a follow-up study by the same group, deletion of the pu element resulted in an effective TRES (inducible) baculovirus system for insect cells (Biotechnol. Prog., 24; 1232-1240). Using the system they demonstrated that inducible overexpression of Lef-2 stimulates polh transcription and its associated recombinant protein expression. However, their system did not demonstrate the capability to down-regulate very late promoter-driven (polh-driven) transcription and its associated protein expression, rather only (further) up-regulation, resulting in a recombinant protein expression from the very late polh promoter exceeding wild type levels. This system therefore appears unable to overcome the problem of high very late promoter driven (essentially constitutive) recombinant protein expression during up-scaling of baculovirus expression and the associated high selective pressure against the expression cassette encoding the recombinant protein, that can resulting in elimination and/or mutation of this cassette.

U.S. Pat. No. 5,939,285 describes the use of a retinoic acid response element (RARE) in a baculovirus promoter (such as polh or P10) to regulate expression of a recombinant protein in insect cells in the presence of a hormone receptor expressed by a gene encoding the same. Such regulation is brought about by varying (between different expression systems) the position and particular arrangement of the RARE/promoter construct relative to the open reading frame encoding the recombinant protein. Such regulation is not brought about by subjecting or exposing any given expression system to a change in conditions.

Dai et al., (Protein Expression and Purification, 2005; 42; 236-245) created an effective, plasmid-based ecdysone receptor transcriptional induction system for use in insect cells. However, the authors did not demonstrate functionality of a comparable inducible system using baculovirus as a vector.

It is therefore an object of the invention to provide a baculovirus system for insect cells in which recombinant protein expression (driven or otherwise controlled) by baculovirus (late and/or very late) promoters is turned “off” during virus amplification and turned “on” only when sufficient recombinant viruses are available for desired expression scale. Without being bound by theory, it is expected that the result of significantly lowered recombinant protein expression during baculovirus amplification is a specific reduction of selective pressure against elimination and/or mutation of (very late promoter-driven) recombinant protein expression cassettes allowing more effective up-scaling and higher overall recombinant protein expression. It is another object of the invention to provide a baculovirus expression system, components thereof and/or methods using same in which insect cells and or baculaorvirus DNA adapted to produce recombinant protein can be increased in number or culture size, for example up to a scale suitable for industrial production of recombinant protein: (i) without, or showing reduced, elimination and/or mutation of the expression cassette encoding the recombinant protein; (ii) without, or showing reduced, loss of yield during such scale up: and/or (iii) with an increase in the overall yield or cost efficiency of recombinant protein production.

### SUMMARY OF THE INVENTION

One or more of the objects of the invention is solved by the baculovirus system, components and kits thereof, and by the methods as described and claimed herein. By virtue of the inducible baculovirus expression system of the present invention, it is now possible to repress recombinant protein expression during amplification of the virus and up-scaling of infected insect cell culture, thereby reducing selection pressure on the virus and the cell for elimination and/or mutation of the expression cassette encoding the recombinant protein. The result is a higher ratio of recombinant protein producing virus to non-producing virus relative to currently available non-inducible baculovirus expression systems. Consequently after induction of recombinant protein expression higher concentrations of recombinant protein per volume of insect cell culture can be achieved. The inducible baculovirus expression system of the present invention is broadly applicable and allows for production of high yields of recombinant protein even in large industrial-scale cell cultures involving many virus passages.

Thus, in one aspect, the invention provides an inducible baculovirus expression system, such as one in insect cells, wherein recombinant protein expression can be repressed during virus amplification in insect cells, comprising: at least one expression cassette A containing a promoter and an open reading frame coding for a controllable transcriptional modulator protein; at least one expression cassette B containing a promoter and an open reading frame coding for a factor which regulates transcriptional activity of a baculovirus late and/or very late promoter (for example VLTF), and in certain embodiment wherein transcriptional activity of the baculovirus late and/or very late promoter decreases with lower than wild type levels of said factor (such as VLTF) in insect cells; at least one expression cassette C containing an open reading frame coding for a recombinant protein under the control of a baculovirus late and/or very late promoter. The system further comprises a transcriptional modulator response element, wherein the controllable transcriptional modulator protein reversibly interacts with said transcriptional modulator response element in one condition, and (reacts/interacts) differently in a second condition, thereby modulating the transcription of expression cassette B or expression cassette C. In certain embodiments, the repressed (off) state, expression level of the recombinant protein of expression cassette C is lower compared to a reference (“wild type”) baculovirus expression system which comprises an expression cassette B containing an open reading frame coding for said VLTF under the control of its original (“wild-type”) promoter, and an expression cassette C as defined above, but does not contain an expression cassette A. Although the response element should have no influence on the expression levels of the recombinant protein in the absence of the expressed product of cassette A, it is preferred that the reference expression system also does not contain the transcriptional modulator response element.

In certain embodiments the inducible baculovirus expression system of the invention further comprises an expression cassette B′ containing an open reading frame coding for said factor (such as VLTF) under the control of a weak promoter producing lower than wild type levels of said factor that still allows baculovirus replication, or containing a promoter and an open reading frame coding for a modified factor (such as a modified VLTF) leading to lower than wild type transcriptional activity of a baculovirus late and/or very late promoter, with the proviso that said modified factor allows baculovirus replication. Preferably, the expression cassette B′ is not inducible.

It will be appreciated that according to the invention the transcriptional modulator protein is a transcriptional repressor protein or a transcriptional activator protein. It will be further understood that if the controllable transcriptional modulator protein is a controllable transcriptional repressor protein, the modulator response element is a transcriptional repressor response element. Alternatively, if the transcriptional modulator protein is a transcriptional activator protein, the modulator response element is a transcriptional activator response element capable of activating transcription of expression cassette B containing an open reading frame coding for said factor (such as VLTF) under the control of a weak promoter producing lower than wild type levels of said factor in its uninduced state. In particular embodiments, the controllable transcriptional modulator protein is a controllable transcriptional repressor protein.

One preferred example for a factor (such as VLTF) according to the invention is vlf-1 or functional homologs thereof.

Typically the inducible baculovirus expression system of the invention is based on the sequence of Autographa californica nuclear polyhedrosis virus (AcMNPV) (Virology 202 (2), 586-605 (1994), whose sequence can currently be retrieved under NCBI Accession No.: NC_{—}001623).

It will further be appreciated that in the above inducible baculovirus expression system the expression cassettes A, B, B′ and/or C can be contained in a transfer vector suitable for fusion with modified baculovirus DNA, in a modified baculovirus DNA, in a genomic baculovirus DNA, in a separate chromosomal DNA within cells infected with the baculovirus, or in a non-chromosomal DNA within a cell infected with a baculovirus.

In a further, related aspect, the invention provides a baculovirus transfer vector, such as one for fusion with (modified) baculovirus DNA, comprising expression cassettes A and C of the inducible baculovirus expression system according to the invention, optionally further comprising a repressor response element in cassette C.

In yet a further aspect, the invention provides a composite baculovirus DNA comprising the inducible baculovirus expression system according to the present invention.

In yet another aspect, the invention provides an intermediate host cell comprising a vector/bacmid containing modified baculovirus DNA comprising expression cassette B of the inducible baculovirus expression system according to the present invention. Expression cassette B may contain an open reading frame coding for said factor (such as VLTF) under the control of a weak promoter and further comprises a transcriptional activator response element. Alternatively, expression cassette B may contain a promoter and an open reading frame coding for said factor and further comprises a transcriptional repressor response element.

Also in respect to this aspect of the invention the vector/bacmid optionally further comprises expression cassette B′ containing an open reading frame coding for said factor (such as VLTF) under the control of a weak promoter producing lower than wild-type levels of said factor allowing baculovirus replication, or containing a promoter and an open reading frame coding for a modified factor (such as a modified VLTF) leading to lower than wild-type transcriptional activity of a baculovirus late and/or very late promoter, and wherein said modified factor allows baculovirus replication.

In a further aspect, the invention provides a method of producing a recombinant protein in insect cells comprising a) introducing the inducible baculovirus expression system or the composite baculovirus DNA as described herein into insect cells, b) culturing said insect cell under conditions where recombinant protein expression is repressed, c) inducing (recombinant) protein production by activating an activator or deactivating a repressor (which in either case is a controllable transcriptional modulator protein), and d) harvesting said recombinant protein. Other methods of producing a recombinant protein in insect cells, as described herein, are also encompassed by the present invention.

In some embodiments the recombinant protein expression is repressed by a transcriptional repressor protein binding to a transcriptional repressor response element in expression cassette C encoding the recombinant protein, or in expression cassette B encoding a late and/or very late transcription factor (such as VLTF) which regulates transcriptional activity of a baculovirus late and/or very late promoter. In other embodiments, the recombinant protein expression is repressed by lack of transcriptional activator protein interaction with a transcriptional activator response element, wherein said interaction activates transcription of expression cassette B containing the open reading frame encoding said factor (such as VLTF) under the control of a weak promoter.

Also encompassed by the present invention is a kit for an inducible baculovirus expression system in insect cells comprising: a) a transfer vector comprising at least one expression cassette C(-) containing a baculovirus late and/or very late promoter, wherein said expression cassette is intended for expressing a recombinant protein under the control of said promoter, b) a modified baculovirus DNA comprising an expression cassette B containing a promoter and an open reading frame encoding a factor which regulates transcriptional activity of a baculovirus late and/or very late promoter (such as VLTF), and c) an expression cassette A encoding a transcriptional repressor protein either on the transfer vector of component a) or on the modified baculovirus DNA of component b). The expression cassette C(-) or B of the kits according to the present invention further comprises a transcriptional repressor response element. In certain of these embodiments, the transcriptional activity of the baculovirus late and/or very late promoter decreases with lower than wild type levels of the factor (such as VLTF) in insect cells, wherein said lower levels of the factor (such as VLTF) still allow viral replication.

Further encompassed by the invention is a kit for an inducible baculovirus expression system in insect cells comprising: a) a transfer vector comprising at least one expression cassette C(-) containing a baculovirus late and/or very late promoter, wherein said expression cassette is intended for expressing a recombinant protein under the control of said promoter, b) a modified baculovirus DNA comprising an expression cassette B containing a transcriptional activator response element, and an open reading frame encoding a late and/or very late transcription factor (such as VLTF) which regulates transcriptional activity of a baculovirus late and/or very late promoter under the control of a weak promoter; and c) an expression cassette A encoding a transcriptional activator protein either on the transfer vector of component a) or on the modified baculovirus DNA of component b). In certain of these embodiments, the weak promoter in cassette B produces lower than wild type levels of the factor (such as VLTF) that still allow viral replication, but cause decreased transcriptional activity of said baculovirus late and/or very late promoter in expression cassette C (i.e., cassette C(-) which further contains the open reading frame for the recombinant protein to be expressed) in insect cells.

In certain embodiments, the above-mentioned kits further comprise on the modified baculovirus DNA an expression cassette B′ containing a) an open reading frame coding for said factor (such as VLTF) under the control of a weak promoter producing lower than wild type levels of said factor allowing baculovirus replication; or b) a promoter and an open reading frame coding for a modified factor (such as a modified VLTF) leading to lower than wild type transcriptional activity of a baculovirus late and/or very late promoter, and wherein said modified factor allows baculovirus replication.

In another aspect, the invention also relates to a recombinant nucleic acid, such as one comprising expression cassette C(-) as described herein, that comprises at least: (i) the promoter of expression cassette C of the inducible baculovirus expression system of the present invention; (ii) a transcriptional modulator response element; and (iii) a cloning site, such as one for inserting, a nucleic acid. In particular embodiments of the recombinant nucleic acids of the invention, said transcriptional modulator response element is not a hormone receptor response element. In other embodiments, said transcriptional modulator response element binds to a bacterial/prokaryotic controllable transcriptional modulator protein and/or is (or is derived from) a bacterial/prokaryotic transcriptional modulator response element. In other particular embodiments the transcription of a nucleic acid inserted into the cloning site is under the control of the promoter in said recombinant nucleic acid.

Also encompassed by the present invention are a vector that comprises and a composition that includes a recombinant nucleic acid of the invention.

Other aspects of the present invention relate to various other methods that use the inducible baculovirus expression system of the present invention, such other methods having various utilities in the scale up of a baculovirus expression system in insect cells. In certain embodiments, after practicing such methods, production of recombinant protein can be induced.

In yet another aspect, the present invention relates to a kit, such as a kit of parts, that includes a plurality of components for the construction and/or use of an inducible baculovirus expression system of the present invention. One embodiment of such a kit comprises at least two components that include (preferably separately): (i) the recombinant nucleic acid, vector, composition or the modified baculovirus DNA of the invention; and (ii) at least one other component for the construction and/or use of an inducible baculovirus expression system, including for example: (a) the expression cassette A as described herein; or (b) instructions describing how to construct and/or use the inducible baculovirus expression system of the present invention, and/or to practice any of the various methods of the present invention. The plurality of components in such a kit may be presented, packaged or stored separately. For example, they may be isolated from one another by being held in separate containers.

Such components, although held separately, may be boxed or otherwise associated together to aid storage and/or transport, and such association may include additional components.

Generally, and by way of brief description, the present invention describes a novel baculovirus protein expression system in insect cells, where the expression of recombinant protein(s) is inducible. By the inducible expression systems of the invention, expression of the recombinant protein of interest can be repressed during virus amplification, and thereafter activated in the presence or absence, respectively, of an inducing molecule or by a change in environmental conditions. The novel expression system does not produce significant levels of recombinant protein by baculovirus-infected cells during virus amplification prior to induction, thereby reducing selection pressure on the expression cassette for the recombinant protein. When induction is initiated, an increased yield of recombinant protein is thus produced relative to presently available, non-inducible baculovirus expression methods. Further, it is demonstrated herein that the yield of protein expression derived from the described methodology further increases relative to non-inducible systems as the scale of protein expression increases.

### BRIEF DESCRIPTION OF THE DRAWINGS

FIG. 1: Illustration of specific loss of recombinant protein expression cassettes during scale-up using baculovirus. Shown is Sal I digested purified baculovirus DNA stained with ethidium bromide after one (lane 1) and three (lane 2) serial viral passages, with the arrow indicating the location of the recombinant protein expression cassette.

FIG. 2: State of the art non-inducible baculovirus expression system. The binding of vlf-1 protein (triangle) encoded by cassette B to its DNA binding site in cassette C is represented by the thin filled line. Recombinant protein production is indicated by the thick arrow (right).

FIG. 3: A) Repressor-based direct induction of recombinant protein transcription: The binding of repressor protein—a controllable transcriptional modulator protein and encoded by expression cassette A—(circle) and VLTF protein—a factor which regulates transcriptional activity of a baculovirus very late promoter and encoded by expression cassette B—(triangle) to their respective DNA binding sites—the transcriptional modulator (repressor) response element, and a baculovirus very late promoter, respectively—are indicated. Binding of the repressor to, in this case, the transcriptional modulator (repressor) response element in the promoter of, cassette C represses recombinant protein production as indicated by the thick arrow. B) Activator-based direct induction of recombinant protein transcription: The binding of activator protein—a controllable transcriptional modulator protein and encoded by expression cassette A—(circle) and a baculovirus transcriptions factor (“BTF”) encoded by expression cassette B—a factor which regulates transcriptional activity of a baculovirus late and/or very late promoter—(triangle) to their respective DNA binding sites—the transcriptional modulator (activator) response element, and a baculovirus late and/or very late promoter, respectively—are indicated. Binding of the activator to, in this case, the transcriptional modulator (activator) response element in the promoter of, cassette C activates (induces) recombinant protein production as indicated by the thick arrow. While other general embodiments of the invention shown schematically in FIGS. 3 to 7 display a “VLTF” as the factor which activates a baculovirus very late promoter, such general embodiments will be understood by the person of ordinary skill, after disclosure of the invention, to also encompass where the factor is a baculovirus transcriptions factor (“BTF”) encoded by expression cassette B—a factor which regulates transcriptional activity of a baculovirus late and/or very late promoter, and the corresponding promoter in expression cassette C may be a baculovirus late and/or very late promoter (as represented in FIG. 3B as “L +/VL Promoter”). In all such general embodiments invention shown schematically in FIGS. 3 to 7, it being further understood by the person of ordinary skill, upon disclosure of the invention, that the transcriptional modulator protein encoded by expression cassette A is a controllable transcriptional modulator protein, and such controllability is used to induce production of recombinant protein (such as in FIG. 3B), or to initially repress, and the induce upon removal of such repression, of recombinant protein (such as in FIG. 3A).

FIG. 4: Activator-based indirect induction of recombinant protein transcription. The binding of activator protein—a controllable transcriptional modulator protein and encoded by expression cassette A—(circle) and VLTF protein—a factor which regulates transcriptional activity of a baculovirus very late promoter and encoded by expression cassette B—(triangle) to their respective DNA binding sites—the transcriptional modulator (repressor) response element, and a baculovirus very late promoter, respectively—are indicated. Binding of the activator to cassette B activates (induces) recombinant protein production as indicated by the thick arrow, by reversibly increasing the levels of available VLTF protein.

FIG. 5: Repressor-based indirect induction of recombinant protein transcription. The binding of repressor protein—a controllable transcriptional modulator protein and encoded by expression cassette A—(circle) and VLTF protein—a factor which regulates transcriptional activity of a baculovirus very late promoter and encoded by expression cassette B—(triangle) to their respective DNA binding sites—the transcriptional modulator (repressor) response element, and a baculovirus very late promoter, respectively—are indicated. Binding of the repressor protein to cassette B represses recombinant protein production as indicated by the thick arrow by reversibly decreasing the levels of available VLTF protein.

FIG. 6: Alternative activator-based indirect induction of recombinant protein transcription. The expression cassettes A, B and C are as described for FIG. 4. Additionally present is expression cassette B′, encoding a VLTF protein that is constitutively expressed and supports baculovirus replication but lower than wild type levels of recombinant protein transcription.

FIG. 7: Alternative repressor-based indirect induction of recombinant protein transcription. The expression cassettes A, B and C are as described for FIG. 5. Additionally present is expression cassette B′, encoding a VLTF protein that is constitutively expressed and supports baculovirus replication but lower than wild type levels of recombinant protein transcription.

FIG. 8: Specific example for a tet repressor-based control of recombinant reporter protein EYFP transcription. Binding of tetracycline repressor (circle) and vlf-1 (triangle) to their respective DNA binding sites is indicated. Binding of the repressor to cassette C represses recombinant protein production as indicated by the thick arrow.

FIG. 9: Generation of composite baculovirus DNA containing expression cassettes A, B, and C as described in FIG. 8. Baculovirus DNA bMON14272 (left) contains expression cassette B encoding native vlf-1 protein. The transfer vector pEXAMPLE1.3 (right) contains expression cassettes A and C encoding the tet repressor protein and EYFP, respectively. In transfer vector pEXAMPLE1.3, direction of transcription 5′ to 3′ for expression cassettes A and C is indicated by arrows. Promoter sequences polh for EYFP and GP64 for tet repressor are indicated as are the DNA binding sites for tet repressor and vlf-1. Fusion of the transfer vector pEXAMPLE1.3 and bMON14272 is carried out by Tn7 transposition (indicated by the dashed cross).

FIG. 10: Transfer vector pEXAMPLE1.1. The EYFP coding sequence driven by the polh promoter is shown, with vlf-1 DNA binding site in polh promoter indicated. Bottom: polh promoter sequence and multiple cloning site from pFBDM.

FIG. 11: Transfer vector pEXAMPLE1.2. The EYFP coding sequence driven by the polh promoter is shown, with vlf-1 DNA binding site in polh promoter, and tet repressor DNA binding site adjacent to polh promoter indicated.

FIG. 12: Transfer vector pEXAMPLE1.3. The EYFP coding sequence under the polh promoter is shown, with vlf-1 DNA binding site in polh promoter, and tet repressor DNA binding site adjacent to polh promoter indicated. A second expression cassette codes for the tet repressor protein and is under control of the GP64 promoter.

FIG. 13: Shows predicted data for a comparison of EYFP expression from inducible pEXAMPLE1.3-bMON14272 (vEXAMPLE1.3) and non-inducible pEXAMPLE1.2-bMON14272 (vEXAMPLE1.2) baculovirus after no virus amplication (left) or two further rounds of virus amplification (right). Baculovirus infected SF21 cells are incubated for 72 hours in the presence, or absence of 5 μg/ml tetracycline (indicated by + or −, bottom). A comparable quantity of cell lysate is compared for absorbance at 510 nm (A₅₁₀). S.E.M (predicted) of three measurements is indicated by dark bars on the top of the graphs.

FIG. 14: Specific example for a tet repressor-based indirect induction of recombinant reporter protein EYFP transcription. Expression cassette B′ produces low levels of native vlf-1 by use of the weak vhspRVvlf1-derived promoter. Binding of tetracycline repressor (circle) and vlf-1 (triangle) to their respective DNA binding sites is indicated. Binding of the tetracycline repressor protein to cassette B represses recombinant protein production indicated by the thick arrow by reversibly decreasing the levels of available vlf-1 protein.

FIG. 15: Generation of composite baculovirus DNA containing expression cassettes A, B, B′ and C as described in FIG. 14. Baculovirus DNA bEXAMPLE2 (left) contains expression cassettes B and B′ which code for the native vlf-1 protein. The transfer vector pEXAMPLE1.3 (right) contains expression cassettes A and C, coding for the tet repressor protein and the EYFP protein, respectively. In transfer vector pEXAMPLE1.3, direction of transcription 5′ to 3′ for expression cassettes A and C is indicated by arrows. Promoter sequences polh for EYFP and GP64 for tet repressor are indicated as are the DNA binding sites for tet repressor and vlf-1. Fusion of the transfer vector pEXAMPLE2.1 and bEXAMPLE2 is carried out by Tn7 transposition (indicated by the dashed cross).

FIG. 16: Strategy to introduce two copies of modified vlf-1 expression cassettes into baculovirus DNA.
A) Plasmid pEXAMPLE2.2 is described schematically. B) A PCR product from pEXAMPLE2.2 (top) containing regions of homology 5′ to native vlf-1 and within the vlf-1 coding region is recombined into native bMON14272 baculovirus DNA (bottom) using ET recombination to create bEXAMPLE2, a baculovirus containing the desired 2 copies of vlf-1.

FIG. 17: Shows predicted data for a comparison of EYFP expression from inducible pEXAMPLE2.1-bEXAMPLE2 (vEXAMPLE2.1) and non-inducible pEXAMPLE1.2-bMON14272 (vEXAMPLE1.2) baculovirus after no virus amplication (left) or two further rounds of virus amplification (right). Baculovirus infected SF21 cells are incubated for 72 hours in the presence, or absence of 5 μg/ml tetracycline (indicated by + or −, bottom). A comparable quantity of cell lysate is compared for A₅₁₀. S.E.M (predicted) of three measurements is indicated by dark bars on. the top of the graphs.

FIG. 18: Specific example for tetracycline repressor-based control of recombinant reporter protein EGFP transcription. Binding of tet-ERFP fusion protein (two circles) and vlf-1 (triangle) to their respective DNA binding sites is indicated. Binding of the repressor to cassette C represses recombinant protein production as indicated by the thick arrow.

FIG. 19: Generation of composite baculovirus DNA containing expression cassettes A, B, and C as described in FIG. 18. Baculovirus DNA bMON14272 (left) contains expression cassette B encoding native vlf-1 protein. The transfer vector pEXAMPLE3.3 (right) contains expression cassettes A and C encoding the tet-ERFP fusion protein and EGFP, respectively. In transfer vector pEXAMPLE3.3, direction of transcription 5′ to 3′ for expression cassettes A and C is indicated by arrows. Promoter sequences polh for EGFP and GP64 for tet-ERFP fusion protein are indicated as are the DNA binding sites for tet-ERFP fusion protein and vlf-1. Fusion of the transfer vector pEXAMPLE3.3 and bMON14272 is carried out by Tn7 transposition (indicated by the dashed cross).

FIG. 20: Transfer vector pEXAMPLE3.1. The EGFP coding sequence driven by the polh promoter is shown, with vlf-1 DNA binding site in polh promoter indicated.

FIG. 21: Transfer vector pEXAMPLE3.2. The EGFP coding sequence driven by the polh promoter is shown, with vlf-1 DNA binding site in polh promoter, and tet repressor DNA binding site adjacent to polh promoter indicated.

FIG. 22: Transfer vector pEXAMPLE3.3. The EGFP coding sequence driven by the polh promoter is shown, with vlf-1 DNA binding site adjacent within the polh promoter, and tet repressor DNA binding site adjacent to the polh promoter also indicated. A second expression cassette codes for the tet-ERFP fusion protein under control of the GP64 promoter.

FIG. 23: Titration of tetracycline levels required for induction of protein expression. Baculovirus bMON14272-pEXAMPLE3.3 from initial transfection was used to infect SF21 insect cells at M.O.I 1.0. in the presence of the indicated levels of tetracycline (A and B, x axis). 72 hours following infection cells were harvested, lysed, and absorption at 490 nm (A, EGFP) and 585 nm (B, ERFP) was measured. S.E.M of three measurements is indicated by dark bars on the top of the graphs.

FIG. 24: Comparison of EGFP expression from virus amplified in the presence or absence of tetracycline. In A) baculovirus bMON14272-pEXAMPLE3.3 was passaged for either two or four virus generations at MOI 0.1 with virus harvested after 48 hours for each passage. Passaging was carried out either in the presence, or absence of 1.0 μg/ml tetracycline (indicated by + or − on x axis). A comparable quantity of cell lysate from each condition was measured for absorbance at 490 nm to quantify relative EGFP expression. S.E.M of three measurements is indicated by dark bars on the top of the graphs. In B) the samples produced as in A) were tested by western blotting using an anti EGFP antibody.

FIG. 25: Specific example for tet repressor-based control of recombinant reporter protein EGFP transcription. The recombinant reporter protein EGFP is driven by a late/very late hybrid promoter. Binding of tet repressor-ERFP fusion protein (two circles) and vlf-1 (triangle) to their respective DNA binding sites is indicated. Binding of the repressor to cassette C represses recombinant protein production as indicated by the thick arrow.

FIG. 26: Comparison of EGFP expression from virus amplified in the presence or absence of tetracycline. In A) composite baculovirus was passaged for either two or four virus generations at MOI 0.1 with virus harvested after 48 hours for each passage. Passaging was carried out either in the presence, or absence of 1.0 μg/ml tetracycline (indicated by + or − on x axis). A comparable quantity of cell lysate from each condition was measured for absorbance at 490 nm to quantify relative EGFP expression. S.E.M of three measurements is indicated by dark bars on the top of the graphs. In B) the samples produced as in A) were tested by western blotting using an anti EGFP antibody.

FIG. 27: Specific example for a tet repressor-based control of recombinant reporter protein EGFP transcription. Binding of tet repressor-ERFP fusion protein (two circles) and vlf-1 (triangle) to their respective DNA binding sites is indicated. Binding of the tet repressor-ERFP fusion protein to the tet repressor DNA binding site present in both cassettes A and C (as second and first, respectively, transcriptional modulatory response elements) represses both tet repressor-ERFP expression, and EGFP expression.

FIG. 28: Comparison of EGFP expression from virus amplified in the presence or absence of tetracycline. In A) composite baculovirus was passaged for either two or four virus generations at MOI 0.1 with virus harvested after 48 hours for each passage. Passaging was carried out either in the presence, or absence of 1.0 μg/ml tetracycline (indicated by + or − on x axis). A comparable quantity of cell lysate from each condition was measured for absorbance at 490 nm to quantify relative EGFP expression. S.E.M of three measurements is indicated by dark bars on the top of the graphs. In B) the samples produced as in A) were tested by western blotting using an anti EGFP antibody.

### DETAILED DESCRIPTION OF THE INVENTION

Provided herein is an inducible baculovirus expression system, such as one for use in insect cells. In one embodiment of this aspect, the invention relates to an inducible baculovirus expression system in insect cells. Any of such embodiments of this aspect may be useful for repressing recombinant protein expression, for example during virus amplification, in insect cells. In alternative such embodiments, recombinant protein expression can be repressed during virus amplification in insect cells.

The inducible baculovirus expression system of the invention comprises:
a) at least one expression cassette A containing a promoter and an open reading frame coding for a controllable transcriptional modulator protein,
b) at least one expression cassette B containing a promoter and an open reading frame coding for a factor which regulates transcriptional activity of a (at least one) baculovirus late and/or very late promoter,
c) at least one expression cassette C containing an open reading frame coding for a recombinant protein under the control of a (at least one) baculovirus late and/or very late promoter, and
d) a transcriptional modulator response element;
wherein said controllable transcriptional modulator protein reversibly interacts with said transcriptional modulator response element in one condition, and (reacts/interacts) differently in a second condition, thereby modulating the transcription of expression cassette B or expression cassette C.

In particular embodiments, the inducible baculovirus expression system of the invention comprises:
a) at least one expression cassette A containing a promoter and an open reading frame coding for a controllable transcriptional repressor protein,
b) at least one expression cassette B containing a promoter and an open reading frame coding for a factor which regulates transcriptional activity of a (at least one) baculovirus late and/or very late promoter,
c) at least one expression cassette C containing an open reading frame coding for a recombinant protein under the control of a (at least one) baculovirus late and/or very late promoter, and
d) a transcriptional repressor response element in expression cassette B or expression cassette C;
wherein said controllable transcriptional repressor protein reversibly interacts with said transcriptional repressor response element in one condition, and (reacts/interacts) differently in a second condition, thereby repressing the transcription of expression cassette B or expression cassette C.

In particular embodiments, the inducible baculovirus expression system of the invention is further characterterised by: (i) said baculovirus late and/or very late promoter is a baculovirus very late promoter and said factor is a factor which regulates transcriptional activity of a baculovirus very late promoter (“VLTF”); and/or (ii) with respect to expression cassette B, transcriptional activity of said baculovirus late and/or very late promoter decreases with lower than wild type levels of said factor in insect cells.

Other embodiments of this aspect of the invention relate to an inducible baculovirus expression system in insect cells, wherein recombinant protein expression (controlled by late and/or very late promoters) can be repressed during virus amplification in insect cells.

By virtue of the inducible baculovirus expression system of the invention it is possible to passage virus with higher levels of non-defective virus (le those able to express recombinant protein) relative to what is achieved by currently available methods.

The term “inducible” as used in the present invention refers to the expression of any protein of interest that is lower in a first condition than in a second. By changing from the first condition to the second condition the protein expression is switched-on or increased. Preferably, this induction is reversible by changing from the second condition back to the first condition, thereby repressing protein expression.

The term “repressed” as used herein refers to the expression of any protein of interest that is higher in a second condition than in a first condition. By changing from the second condition to the first condition the protein expression is switched-off or decreased. Preferably, this repression is reversible by changing from the first condition back to the second condition, thereby inducing protein expression.

As used herein “baculovirus expression system” refers to a system using baculoviruses coding for a recombinant protein allowing production of said recombinant protein in insect cells, particularly insect cell lines. A baculovirus expression system generally comprises all elements necessary to achieve recombinant protein expression in insect cells. Thus, such a system may comprise a transfer vector, a modified baculovirus DNA and optionally a helper plasmid or a modified insect cell, wherein the transfer vector can be fused to the modified baculovirus DNA to form a composite baculovirus. The composite baculovirus DNA generally comprises all components of the baculovirus expression system. In certain embodiments however, the composite baculovirus DNA may lack one or other such components which are then comprised in a modified cell line that forms part of the expression system. Most of the presently used baculovirus expression systems are based on the sequence of Autographa californica nuclear polyhedrosis virus (AcMNPV) ((Virology 202 (2), 586-605 (1994), NCBI Accession No.: NC_{—}001623).

The term “virus amplification” as used herein refers to the propagation of virus and in particular to serially passaging a virus to scale up number of virus particles starting from a clonal virus. Such propagation is typically conducted using, such as during culture of, insect cells.

In accordance with the present invention the inducible baculovirus expression system comprises at least one expression cassette A, at least one expression cassette B and at least one expression cassette C and a transcriptional modulator response element reversibly interacting with a controllable transcriptional modulator protein in one condition, and (reacting/interacting) differently in a second condition, thereby modulating expression of expression cassette B or expression cassette C.

For the purposes of the present invention, “(responds/interacts) differently”, with respect to controllable the transcriptional modulator protein, includes the meaning of where said modulator protein maintains a particular occupancy on the transcriptional modulator response element in one condition, and a lower occupancy on said response element in a second condition. For example, in one condition the binding constant of said modulator protein changes such that its effective occupancy on said response element is lower, and hence said modulator protein becomes (far) more likely to dissociate from, or not to associate with, said response element, including to such an extent that said modulator protein can be considered to no longer (effectively) interact or bind to said response element, and in particular to no longer (effectively) modulate the transcription expression cassette B or expression cassette C. In certain embodiments of the present invention, the modulation of transcription of expression cassette B or expression cassette C, upon interaction of the controllable transcriptional modulator protein. is modulated in one condition relative to the second condition. In other embodiments of the present invention, the modulation of transcription of expression cassette B or expression cassette C is in, functions in, occurs in, can function in or can occur in, insect cells.

As used herein the term “expression cassette” refers to an entity made up of a gene and the sequences controlling its expression. An expression cassette comprises at least a promoter sequence, an open reading frame encoding for a protein, and a 3′ untranslated region. The expression cassette can be part of a vector DNA, plasmid DNA, bacmid DNA, genomic DNA, a virus, a cell or any other DNA/RNA or DNA/RNA containing organism.

The expression cassette A of the present invention contains a promoter and an open reading frame coding for a controllable transcriptional modulator protein.

The term “promoter” as used herein is a region of DNA that facilitates the transcription of a particular gene. Promoters are typically located adjacent to the genes they regulate, on the same strand and upstream (towards the 5′ region of the sense strand).

The term “open reading frame” (ORF) as used herein is a portion of an organism's genome which contains a DNA sequence that could potentially encode a protein. In a gene, ORFs are located between the start-code sequence (initiation codon) and the stop-code sequence (termination codon).

A “controllable transcriptional modulator” as used herein can be a controllable transcriptional activator or a controllable transcriptional repressor, activating or repressing transcription, respectively, upon reversibly interacting with the respective transcriptional modulator response element. The reversible interaction, e.g., reversible binding, can be controlled by the presence of an inducer molecule or by a change in environmental conditions. The DNA bound transcriptional activator typically activates transcription through interaction with the basal transcription machinery or with other factors that upregulate transcription. The DNA bound transcriptional repressor typically sterically inhibits transcription initiation or transcription elongation.

The term “transcriptional modulator response element” as used herein is a consensus DNA binding site for a controllable transcriptional modulator, typically, but not necessarily located in or adjacent to promoters. The transcriptional modulator response element is a transcriptional activator response element in case of a controllable transcriptional activator and a transcriptional repressor response element in the case of a controllable transcriptional repressor.

Preferably, in the present invention the transcriptional modulator response element is located in an expression cassette, activating or repressing transcription of the same expression cassette. In principle, the transcriptional modulator response element can be located anywhere within the expression cassette, such as e.g., in the 5′ untranslated region, in the promoter region, between the promoter and the transcription initiation site, within the open reading frame or in the 3′ untranslated region of the expression cassette. It is also possible that the transcriptional modulator response element reversibly interacting with the controllable transcriptional modulator is located outside that same expression cassette, as long as said interaction activates transcription of said expression cassette. A non-limiting example would thus be a transcriptional repressor response element within a polh enhancer-like element to repress polh driven transcription from expression cassette C.

In certain embodiments of the present invention, the transcriptional modulator response element is comprised in or in proximity to (adjacent to) the promoter of said expression cassette in expression cassette B or expression cassette C. For the purposes of the present invention, a transcriptional modulator response element is considered “in proximity” (or adjacent) to a promoter if it is situated downstream or upstream of said promoter at a distance of between 10 and 1,000 bp, and preferably less than 100 bp, from the transcription start-site of said promoter.

In particular embodiments of the present invention, the transcriptional modulator response element is a transcriptional repressor response element in expression cassette C, preferably in or in proximity to (adjacent to) the promoter of expression cassette C.

The expression cassette B of the present invention contains a promoter and an open reading frame coding for a factor which regulates transcriptional activity of a baculovirus late and/or very late promoter, and in certain embodiments (including such as when the controllable transcriptional modulator is a controllable transcriptional activator) wherein transcriptional activity of the baculovirus late/and or very late promoter decreases with lower than wild type levels of said factor in insect cells.

In other certain embodiments of the present invention, the expression cassette B is native to and/or is found in the genome of a wild-type baculovirus.

In certain embodiments of the present invention, said factor that regulates transcriptional activity of the baculovirus (late and/or very late) promoter stimulates, can stimulate and/or is capable of stimulating transcriptional activity of said promoter.

In particular embodiments of the present invention, said factor is one which regulates transcriptional activity of a baculovirus very late promoter (referred to herein as “VLTF”), such as where transcriptional activity of the baculovirus very late promoter decreases with lower than wild type levels of said VLTF in insect cells.

The factor (eg VLTF) can be a naturally occurring factor (such as VLTF)—ie it can be considered a native factor such as one expressed by the expression cassette B that is native or naturally occurring to the baculovirus system—or the factor may be a transcriptionally functional homolog thereof.

According to some embodiments of the present invention, expression from expression cassette B can be directly regulated by a controllable transcriptional modulator interacting with a transcriptional modulator response element, wherein the transcriptional modulator response element is preferably inside the expression cassette B. In the latter case the native factor of the baculovirus expression system is, in certain embodiments, replaced by a factor expressed by expression cassette B. In other embodiments, a native transcription factor may be modified or its transcription may be modified, such as described herein, so that its transcription regulatory activity is reduced.

In alternative embodiments of the present invention, the factor encoded by expression cassette B and the baculovirus late and/or very late promoter (in expression cassette C) that it regulates come from a different baculovirus species or strain to the baculovirus that form the other parts of the inducible baculovirus expression system, such that any native transcription factors expressed by the other parts of the inducible baculovirus expression system do not regulate the transcriptional activity of said promoter, and its transcriptional activity is (predominately) modulated by the factor encoded by expression cassette B, and (predominately) not by any native transcription factors expressed by the other parts of the inducible baculovirus expression system.

The term “late promoter” as used herein is a promoter used for baculovirus late gene expression, for example to express genes between about 6 and 24 hours post infection, such as between about 12 and 18 hours post infection. The term “very late promoter” as used herein is a promoter used for baculovirus very late gene expression, for example to express genes between about 18 and 72 hours post infection, such as between about 18 and 24 or about 24 and 72 hours post infection. Two highly expressed very late genes have been characterized, the polyhedrin and the p10 gene, and their respective very late promoters have been named polh and p10 promoter. Examples for AcMNPV-derived sequences serving as very late promoters are given as Seq ID No: 1 (polh) and Seq ID No: 2 (p10). During the very late phase of infection both genes undergo a burst of transcription, leading to accumulation of their respective RNAs and proteins in the cell (Virology 248, 131-138). Very late promoters are thought to differ from late promoters primarily by the presence of a burst sequence, a sequence downstream of the transcription start site that is 90% A and T (J. Virol. 79, 1958-1960).

As will be appreciated by the person of ordinary skill, the term “baculovirus late and/or very late promoter” comprises any promoter that functions as a late and/or functions as a very late promoter for a baculovirus, and includes such promoters that comprise nucleic sequences derived from a late, or from a very late, promoter that is present in the genome of a wild type baculovirus. In this context, “derived from” includes nucleic acids sequences that are greater than 80%, such as 85%, 90%, 95%, 99% or 100% identical over about 5, 10, 15, 20, between 20 and 30 or between 30 and about 50 bp. Accordingly, synthetic promoters, that are not native to a given baculovirus species—such as hybrid promoters that contain fragments of both the late Pcap promoter (Gene. 91 (1990) 87-94) and a very late promoter such as polh or p10. Such hybrid promoters may be activated by baculovirus factors (such as those encoded by expression cassette B) at both late and very late stages of infection.

The term “a factor which regulates transcriptional activity of a baculovirus late and/or very late promoter”, (such as, when referring to a very late promotor, abbreviated “VLTF” herein), refers to any molecule, such as a peptide, polypeptide or protein, capable of regulating transcriptional activity of a baculovirus late and/or very late promoter, and in certain embodiments (including such as when the controllable transcriptional modulator is a controllable transcriptional activator) wherein its transcriptional activity decreases with lower than wild type levels of the factor in insect cells. One example for a suitable factor according to the present invention is vlf-1.

In preferred embodiments of the invention, the factor is vlf-1 of Seq ID No:3 (encoded for example by Seq ID No: 4 or Seq ID No: 10) or a transcriptionally functional homolog thereof. Vlf-1 interacts with the burst sequence of p10 and polyhedrin regulatory regions and selectively up-regulates genes under the control of the very late promoters.

Other baculovirus proteins that may act as a factor (like a VLTF) include FP25, ie-1, Lef-2, Lef-4, PK-1, Ac43, polh enhancer-like binding proteins, or any functional homologs of the above proteins; and in certain embodiments (including such as when the controllable transcriptional modulator is a controllable transcriptional activator), provided that lower levels of such a factor (like a VLTF) lead to lower than wild type levels of recombinant protein expression from expression cassette C in insect cells while still allowing virus replication.

Yet other baculovirus proteins that may act as a suitable factor in the present invention (in this case that regulate transcription of a baculovirus late promoter) include Lef-1, Lef-2, Lef-3, Lef-4, Lef-5, Lef-6, Lef-7, Lef-8, Lef-9, Lef-10, Lef-11, Lef-12, IE-1, IE-2, Ac69, Ac38, Ac36, p47, p143, p35, DNAPOL, HCF-1. Further information on such factors can be found in J. Virology, 27: 10197-10206. It is known by the person of ordinary skill that some factors (such as Lef-2 and Lef-4) regulate baculovirus late and also regulate very late promoters.

Methods for determining if a factor regulates transcriptional activity of a baculovirus late and/or very late promoter have been described, such as exemplarily for vlf-1 by Yang and Miller (Virology 248, 131-138). Briefly, the gene of interest is mutated, deleted or, if the gene is essential for replication, cloned under the control of a weak promoter. The weak promoter can be any weak promoter described in the literature for the respective factor or can be generated as described for vlf-1 accordingly, for example by inserting a DNA fragment containing the hsp70 promoter between the factor open reading frame and its original promoter in opposite orientation to the original promoter. In case the gene of interest is essential for the replication of baculovirus, the viability of the virus indicates that sufficient amounts of the gene are produced by the recombinant virus. Amounts of the potential factor and polyhedrin-driven (or another late and/or very late promoter) expression can be easily monitored in cells infected with baculovirus with the gene of interest mutated or deleted or under the control of a weak promoter and compared to cells infected with “wild type” baculovirus at the same M.O.I. by, e.g., immunoblot examination. The person skilled in the art will appreciate that any other method for detecting quantitatively or semi-quantitatively the product of the gene of interest and the late and/or very late gene product (for example polyhedrin or alternatively p10) can be applied. Detection can be at the transcriptional or translational level, detecting mRNA or protein levels, respectively. Non-limiting examples for detection methods are flow cytometry, microscopy, real-time PCR, immuno- or Western blotting, ELISA and Northern blotting. Further, the polyhedrin or, alternatively, p10 gene or any suitable late and/or very late baculovirus gene, can be replaced by a reporter gene such as a genes encoding chloramphenicol acetyltransferase (CAT), a fluorescent protein like GFP, YFP or their enhanced analogues, a luminescent protein like luciferase or any other protein that is easily detectable. Methods for detecting and quantifying reporter gene expression are well known in the art.

The term “wild type virus” as used herein refers to the phenotype of the typical form of a species as it occurs in nature including expression systems derived therefrom. In the case of AcMNPV the wild type virus is encoded by the sequence of NCBI accession number NC_{—}001623 (Virology 202 (2), 586-605 (1994)) and wild type expression systems are based on this sequence.

As used herein a “wild type expression system” is not inducible (and/or repressible), meaning that expression of a protein under the control of a very late promoter is not controlled by an engineered system of transcriptional repressor or activator proteins and their respective response elements, neither directly nor indirectly via any factors such as baculovirus (or non-baculovirus) transcription factors acting on late and/or very late promoters, particularly a factor such as vlf-1. Additionally the factor (such as VLTF) is full-length and not modified in a way that interferes with its transcriptional activity, potential virus replication activity or stability of mRNA or protein and is expressed under the control of its original promoter. Described in terms of expression cassettes as defined herein a wild type expression system does not contain an expression cassette A and/or its corresponding transcriptional modulator response element as defined herein, but contains an expression cassette B with an open reading frame coding for a factor (such as VLTF) under the control of a promoter and an expression cassette C as defined herein, wherein the factor (such as VLTF) of expression cassette B is full-length and not modified in a way that interferes with its transcriptional activity. potential virus replication activity or stability of mRNA or protein, and the respective promoter is the original promoter or a functional homolog thereof.

The term “wild type levels of the factor” means levels expressed by an expression cassette in a “wild type” baculovirus expression system as defined above, i.e. comprising an open reading frame coding for said factor (such as VLTF) under the control of its original promoter, wherein the factor (such as VLTF) of expression cassette B is full-length and not modified in a way that interferes with its transcriptional activity, potential virus replication activity or stability of mRNA or protein.

The term “lower than wild type levels of factor” as used herein refers to a factor expression level in a baculovirus expression system that is relatively lower than the “wild type levels of the factor”. The level of factor (such as VLTF) expression in a baculovirus system can be lowered by several means. Non-limiting examples would be to express the factor (such as VLTF) under the control of a weak promoter as described above, or by any mutation that decreases factor (such as VLTF) mRNA or protein levels. Levels of factor (such as VLTF) expression in insect cells comprising such a baculovirus expression system can be directly compared to factor (such as VLTF) levels expressed in insect cells infected with “wild type virus” or a wild type baculovirus expression system. Quantitative or semi-quantitative detection of factor (such as VLTF) can for example be achieved at the translational level detecting factor (such as VLTF) protein. Non-limiting examples for protein detection methods are flow cytometry, microscopy, immuno- or Western blotting and ELISA. It is well known in the art that detection of proteins can be simplified in many cases by expressing the protein containing a tag at the C- or N-terminus, such as a Flag-tag or a His-tag, that is recognized by commercially available monoclonal or polyclonal antibodies. This is particularly useful in cases where no antibodies recognizing the protein are known or available. As the tags are small they usually do not interfere with the function of the protein. In some cases quantitative or semi-quantitative detection of factor (such as VLTF) can also be achieved at the transcriptional level, detecting factor (such as VLTF) RNA or mRNA levels. Non-limiting examples for suitable detection methods are RT-PCR, real-time PCR and Northern blotting.

Although in certain embodiments of the inducible baculovirus expression system of the present invention any lower than wild type expression level of the factor (such as VLTF) may in principle be sufficient to be effective in reducing expression levels from expression cassette C, it is preferred that factor (such as VLTF) protein levels in such systems are less than about 75%, more preferably less than 50%, 40% or even 30% of wild type levels.

The abbreviation M.O.I as used herein refers to the multiplicity of infection and is the ratio of infectious virus particles to infection targets (e.g. cells). For example, when referring to a group of cells inoculated with infectious virus particles, the multiplicity of infection or M.O.I is the ratio defined by the number of infectious virus particles deposited in a well divided by the number of target cells present in that well.

The term “transcriptionally functional homolog” as used herein relates to a protein factor (such as VLTF) that does not have the same amino acid sequence than the protein factor (such as VLTF) it refers to, but is functionally identical or similar in its transcriptional activation of a baculovirus late and/or very late promoter. This means that the protein factor (such as VLTF) can be replaced with its transcriptionally functional homolog in a recombinant baculovirus expression system without any significant changes in expression levels of the recombinant protein under the control of the late and/or very late promoter. No significant changes in this context means that the transcriptional activity of a very late promoter in the presence of the transcriptionally functional homolog should be at least 80% of transcriptional activity of the wild type factor (such as VLTF), preferably 85%, 90%. 95%, 100% or even more than 100% the transcriptional activity of the wild type factor (such as VLTF). This includes fragments of the full-length protein factor (such as VLTF) with a transcription activity from a very late promoter similar to wild type full-length protein factor (such as VLTF).

The expression cassette C of the present invention contains an open reading frame coding for a recombinant protein under the control of a baculovirus late and/or very late promoter. In certain embodiments expression cassette C further comprises a transcriptional modulator response element functionally interacting with the expression product of expression cassette A which, in certain embodiments, is a transcriptional repressor response element.

According to the present invention, expression of the recombinant protein expressed by expression cassette C can be directly or indirectly repressed, and in certain embodiments wherein, in the repressed (off) state, expression levels of said recombinant protein of (encoded by) expression cassette C is lower as compared to a non-inducible baculovirus expression system with an expression cassette B containing an open reading frame coding for said factor (such as VLTF) under the control of its original promoter, and an expression cassette C as defined above, but without an expression cassette A and/or its corresponding transcriptional modulator response element. Preferably, according to the present invention, transcription from late and/or very late promoters in expression cassette C in the repressed (off) state should be less than about 50% of wild type levels, more preferably less than 30%, 20% or even less than 10% of wild type levels. In fact, in some systems, even larger repression factors may be achieved, particularly if the timing of the repressor or activator protein expression is synchronized with the expression from cassettes B and/or C.

The term “recombinant protein” as used herein refers to any protein not naturally expressed under the control of a baculovirus late and/or very late promoter. In view of the inducible nature of the baculovirus expression systems provided herein, the recombinant protein can also be a “toxic” protein for the host cell. Further the DNA sequence encoding the recombinant protein can be a naturally existing DNA sequence or a non-natural DNA sequence. The recombinant protein can be modified in any way. Non-limiting examples for modifications can be insertion or deletion of post-translational modification sites, insertion or deletion of targeting signals, fusion to tags, proteins or protein fragments facilitating purification or detection, mutations affecting changes in stability or changes in solubility or any other modification known in the art. In certain embodiments of the present invention, the recombinant protein is a virus-like partial (VLP), including a VLP for use in vaccination of humans or animals such as for vaccination against influenza.

The expression system of the invention can further comprise at least one expression cassette B′ expressing a factor (such VLTF) with deficient transcriptional activity, but allowing baculovirus replication. In some embodiments of the invention the expression cassette B′ contains an open reading frame coding for a factor (such as VLTF) under the control of a weak promoter producing lower than wild type levels of said factor (such as VLTF) that still allow baculovirus replication but are not sufficient for wild type levels of very late transcription. In other embodiments, the expression cassette B′ contains a promoter and an open reading frame coding for a modified factor (such as modified VLTF) leading to lower than wild type transcriptional activity of a baculovirus late and/or very late promoter, and further wherein said modified factor (such as modified VLTF) still allows baculovirus replication.

The term “allowing replication” as used herein refers to viruses that are replication competent. If a protein is essential for replication, deletion of said protein results in a recombinant baculovirus that is not replication competent. Similarly a mutation or any other modification of a protein essential for replication may result in a recombinant baculovirus that is no longer replication competent. Consequently the protein essential for replication is expressed at sufficient levels to yield a recombinant baculovirus that is replication competent. Alternatively, if the protein is modified, the modification does not interfere with the protein's ability to support replication. It is generally accepted in the art that recombinant baculoviruses are no longer considered replication competent if they produce titers on the order of 10⁵ plaque-forming units (PFU)/ml or less in insect cell supernatants following initial transfection, or if they are no longer rescuable following clonal selection (Virology 245, 99-109). A PFU is a measure of the number of particles capable of forming plaques per unit volume. It is a functional measurement rather than a measurement of the absolute quantity of particles: viral particles that are defective or which fail to infect their target cell will not produce a plaque and thus will not be counted. Baculovirus infected cells, stop dividing and grow in size. Therefore titers of infectious baculovirus formed upon the initial transfection mixture can also be measured by determining the quantity of virus stock required to immediately stop cell division, or by measuring the ratio of enlarged (infected) versus normal (uninfected) cells. As replication is a prerequisite for up-scaling of recombinant baculovirus expression, any recombinant baculovirus that can be amplified is considered replication competent, wherein amplified means growing virus from a small starting clonal pool. Methods for determining baculovirus titer, and methods for clonal selection are well established and known in the art (O'Reilly et al. Oxford University Press, New York, 1994)

It is an advantage in certain embodiments of the invention that a factor (such as VLTF) can be expressed independently from expression cassette B′ to allow baculovirus replication, particularly in cases where the factor (such as VLTF) of expression cassette B is essential for baculovirus replication and expression of the factor (such as VLTF) from expression cassette B can be repressed to levels that no longer allow baculovirus replication. In other words the factor (such as VLTF) expressed by expression cassette B′ can rescue the virus that would otherwise not be viable with factor (such as VLTF) expressed by expression cassette B alone in the repressed state.

The term “modified factor” refers to any factor (such as VLTF) leading to lower than wild type transcriptional activity of a baculovirus late and/or very late promoter but still allowing baculovirus replication. The modification can include one or more amino acid deletion(s), insertion(s), or substitution(s), or post translational modifications. Modifications leading to a diminished transcriptional activity of the factor (such as VLTF) from a baculovirus late and/or very late promoter but still allowing replication are either known in the art or can be conveniently determined by those skilled in the art by assessing transcriptional activity or replication as described above. Transcriptional activity can further be determined in transient transfection assays in insect cells as described by Todd et al. (Journal of Virology, 70; 2307-2317) and Yang and Miller (Virology, 245; 99-109). In addition to the description above detailed methods to analyze baculovirus replication are well established in the art (see O'Reilly et al. Oxford University Press, New York, 1994). In preferred embodiments, the modified factor (such as a modified VLTF) exhibits a transcriptional activity that is less than about 75%, and more preferably less than 50%, 40%, or even 30% compared to “wild type” factor (i.e., the native factor under the control of its original promoter). In certain embodiments the modified factor (such as modified VLTF) is a modified vlf-1. Examples of modified vlf-1 proteins in accordance with the present invention are vlf-1 of Seq ID No:3, but carrying a C202Y mutation or a cysteine insertion between P23 and R24 (vcBsuSsevlf1, Virology 248, 131-138). In other embodiments, the modified factor is one modified from a factor that regulates transcription of a baculovirus late promoter.

Although the factor (such as VLTF) encoded in expression cassettes B and B′ must not necessarily be the same (which in this context includes modified versions of the same factor), typically the factor or modified factor expressed by expression cassette B′ will indeed be the same factor as expressed by expression cassette B. Preferably, the factor of expression cassette B and the factor or modified factor of expression cassette B′ are vlf-1 (including modified versions thereof). Alternatively, in the absence of wild type levels of vlf-1, the factor expressed at lower than wild type levels or the modified factor expressed by expression cassette B′ can be vlf-1 or modified vlf-1, respectively and the factor expressed by expression cassette B can be a factor different from vlf-1, e.g., FP25, ie-1, Lef-2, Lef-4, PK-1, Ac43, or polh enhancer-like binding proteins, or a factor that regulates transcription of a baculovirus late promoter; and in certain embodiments (including such as when the controllable transcriptional modulator is a controllable transcriptional activator), provided that lower levels of such a factor (like a VLTF) or modified variants thereof still allow virus replication, but lead to lower than wild type transcriptional activity of a baculovirus late and/or very late promoter in expression cassette C in insect cells.

As mentioned earlier herein, expression of expression cassette B can be directly regulated by a controllable transcriptional modulator interacting with its respective modulator response element. In certain embodiments the transcriptional modulator is a transcriptional activator protein and expression cassette B contains an open reading frame coding for the factor (such as VLTF) under the control of a weak promoter producing lower than wild type levels of factor allowing baculovirus replication, preferably further comprising a transcription activator response element.

As used herein the term “weak promoter” refers to any promoter producing “lower than wild type levels of factor” as defined herein. An example for a weak promoter for vlf-1 of Seq ID No: 9 derived from the vhspRVvlf1 construct described in (Virology 248, 131-138). The weak promoter derived from vhspRVvlf1 was created by inserting a fragment from the Drosophila melonogaster HSP70 heat shock protein promoter 3 bps upstream of the vlf-1 translation start codon in opposite direction to the vlf-1 promoter. This method can be used accordingly for any factor to generate a weak promoter from its original promoter. Any non-native factor promoter sequence that results in lower transcription of said factor than wild-type levels can likewise be used as a weak promoter in accordance with the present invention. Preferably, the weak promoter produces less than about 75%, and more preferably less than 50%, 40%, or even 30% of the factor (such as VLTF) compared to “wild type” factor (i.e., under the control of its original promoter).

In preferred embodiments the promoter in expression cassettes B and B′, if it is not defined as a weak promoter, is the original promoter of the respective factor (such as VLTF) or a functional homolog thereof, wherein functional homolog means that the transcriptional activity is generally close to that of the original promoter. The transcriptional activity is preferably at least 80%, more preferably at least 85%, 90%, 95%, 100% or even more than 100% of that of the original promoter of the factor (such as VLTF).

According to the present invention the expression cassettes described herein can be contained in a transfer vector suitable for fusion with modified baculovirus DNA, in a modified baculovirus DNA, in a genomic baculovirus DNA, in a separate chromosomal DNA within cells infected with the baculovirus, or in a non-chromosomal DNA within a cell infected with a baculovirus.

Thus, the invention also provides a baculovirus transfer vector wherein the transfer vector comprises expression cassettes A and C of the inducible baculovirus expression system as described herein. In some embodiments of this aspect of the invention the expression cassette C further comprises a (transcriptional) repressor response element and/or expression cassette A encodes a controllable transcriptional repressor protein. In other embodiments, the transfer vector may further comprise an expression cassette B′ and/or an expression cassette B; and in certain embodiments provided the modified baculovirus DNA does not contain the gene encoding its native factor (such as VLTF) regulating transcriptional activity of the very late promoter. In certain embodiments of this aspect of the invention, the baculovirus transfer vector is for fusion with baculovirus DNA, including for fusion with modified baculovirus DNA.

The invention also provides a composite baculovirus DNA comprising the inducible baculovirus expression system as described herein.

In another aspect, the invention provides an intermediate host cell comprising a vector/bacmid containing modified baculovirus DNA comprising expression cassette B of the inducible baculovirus expression system described herein. In certain embodiments the expression cassette B contains an open reading frame coding for a factor (such as VLTF) as defined herein under the control of a weak promoter and further comprises a transcriptional activator response element. In other embodiments the expression cassette B comprises a promoter, an open reading frame coding for a factor (such as VLTF) and a transcriptional repressor response element. The intermediate host cell of the invention can further comprise at least one expression cassette B′ on the vector/bacmid containing a promoter and an open reading frame coding for a factor (such as VLTF), wherein either the promoter is a weak promoter producing low levels of said factor, or said factor is a modified factor (such as a modified VLTF), which in both instances lead to lower than wild type transcriptional activity of a baculovirus late and/or very late promoter, while still allowing baculovirus replication. In certain embodiments of the present invention, the weak promoter and/or the transcriptional modulator response element of expression cassette B is heterologous to the open reading frame for the factor encoded by said expression cassette.

The intermediate host cell described herein is any one of a bacterial cell, a yeast cell, a mammalian cell or an insect cell, preferably a bacterial cell or an insect cell, more preferably a bacterial cell.

The present invention also provides an insect cell comprising the inducible baculovirus expression system or the composite baculovirus DNA as described herein.

Further, the invention provides a method of producing a recombinant protein in insect cells comprising the steps of a) introducing the inducible baculovirus expression system or the composite baculovirus DNA of the invention into insect cells, b) culturing said insect cell allowing viral amplification under conditions where recombinant protein expression (controlled by a baculovirus late and/or very late promoter) is repressed, c) inducing recombinant protein production by activating an activator or deactivating a repressor (which in either case is the controllable transcriptional modulator protein of the inducible baculovirus expression system of the invention); and d) harvesting said recombinant protein.

The term “introducing” as used herein refers to any method known to the person skilled in the art to bring the DNA encoding the inducible baculovirus expression system or the baculovirus comprising the DNA encoding the inducible baculovirus expression system into an insect cell. This comprises methods such as transfection, microinjection, transduction and infection. Methods for transfecting DNA into insect cells are known to the person skilled in the art and can be carried out, e.g., using calcium phosphate or dextran, by electroporation, nucleofection or by lipofection.

In the inducible baculovirus expression systems of the invention recombinant genes are cloned into the baculovirus genome either through recombination of modified baculovirus DNA and recombinant gene-containing plasmids in insect or bacteria cells, or through in vitro generation. Using any of these methods, sufficient recombinant protein-encoding viruses or DNA are generated to infect or transfect, respectively, insect cell culture volumes in the order of a few milliliters.

In a related aspect, the present invention also relates to a method of producing a recombinant protein in insect cells, said method comprising steps b) to d) of the aforementioned method, wherein the insect cells cultured in step b) are insect cells that comprise an inducible baculovirus expression system of the invention, or are insect cells that comprise the composite baculovirus DNA of the invention.

The term “culturing” as used herein refers to maintaining insect cells in a suitable medium and under conditions allowing the cell to be maintained for the virus to amplify. This also includes up-scaling of virus producing insect cell cultures, comprising serial steps of harvesting virus and reinfecting larger insect cell cultures, or comprising a single step of infecting a cell culture at low M.O.I and allowing the multiple generations of baculoviral passage to occur in situ by maintaining the culture in log phase cell densities. In certain embodiments culturing comprises the generation of 10, 100, 1000 or even more liters of insect cell culture comprising the inducible baculovirus expression system of the invention.

In accordance with the methods provided by the invention, recombinant protein expression is repressed during “culturing” the insect cells and amplification of virus comprising the expression system of the invention. In one embodiment the recombinant protein expression is repressed by a transcriptional repressor protein binding to a transcriptional repressor response element thereby repressing expression of expression cassette C encoding the recombinant protein or expression of expression cassette B encoding a late and/or very late transcription factor (such as “VLTF”) which regulates transcriptional activity of a baculovirus late and/or very late promoter. In another embodiment recombinant protein expression is repressed by lack of transcriptional activator protein interaction with a transcriptional activator response element, including certain embodiments wherein such interaction activates transcription of expression cassette B containing the open reading frame encoding said factor (such as a VLTF) under the control of a weak promoter.

The term “inducing protein production” as used herein refers to changing from a first condition to a second condition thereby switching-on or increasing recombinant protein expression, wherein the change in condition can be achieved by addition or deprival of a chemical inducer molecule or corepressor, or by a change of environmental factors such as light or temperature.

The insect cell according to the present invention, including an intermediate host insect cell, can be any insect cell supporting baculovirus production. Examples for insect cells supporting baculovirus production are cells derived from Spodoptera frugiperda, Trichoplusia ni, Plutella xylostella, Manduca sexta, and Mamestra brassicae. Preferred insect cells in the context of the invention are the IPLB-SF21AE cell or its clonal isolate, the Sf9 cell.

In yet another aspect the invention provides a kit for an inducible baculovirus expression system, such as in, or for use in, insect cells, comprising two or more of the components of the inducible expression system of the invention on a transfer vector and a modified baculovirus DNA. The modified baculovirus DNA can be provided in any suitable form, for example as a DNA in a purified form, in a bacterial cell, in a yeast cell, or in an insect cell. In some embodiments of the kit, the transfer vector comprises at least one expression cassette C(-) into which an open reading frame encoding a recombinant protein can be cloned and wherein the expression of said recombinant protein is under the control of a baculovirus late and/or very late promoter, and further comprises a modified baculovirus DNA comprising an expression cassette B containing a promoter and an open reading frame encoding a factor which regulates transcriptional activity of a baculovirus late and/or very late promoter (such as a VLTF); and in certain embodiments wherein transcriptional activity of the baculovirus late and/or very late promoter decreases with lower than wild type levels of said factor in insect cells. In some embodiments, the kit further comprises an expression cassette A encoding a transcriptional repressor protein, which can be located on the transfer vector together with expression cassette C(-) or on the modified baculovirus DNA together with the expression cassette B. The transcriptional repressor response element is located within expression cassette B or C(-).

In other embodiments, the kit contains the transfer vector comprising at least one expression cassette C(-) into which an open reading frame encoding a recombinant protein can be cloned and wherein the expression of said recombinant protein is under the control of a baculovirus late and/or very late promoter, and further comprises a modified baculovirus DNA comprising an expression cassette B containing a transcriptional activator response element, an open reading frame encoding a late and/or very late transcription factor (such as a VLTF) which regulates transcriptional activity of a baculovirus late and/or very late promoter under the control of a weak promoter, wherein the weak promoter produces lower than wild type levels of said factor allowing viral replication, but in certain embodiment causing decreased transcriptional activity of said baculovirus late and/or very late promoter in expression cassette C in insect cells. Such kits further comprise an expression cassette A encoding a transcriptional activator protein, which can be located on the transfer vector together with expression cassette C(-) or on the modified baculovirus DNA together with the expression cassette B.

The modified baculovirus DNA of the kits of the invention can further comprise an expression cassette B′. Expression cassette B′ contains an open reading frame coding for a factor (such as VLTF) under the control of a weak promoter producing lower than wild type levels of said factor allowing baculovirus replication or a promoter and an open reading frame coding for a modified factor (such as a modified VLTF) leading to lower than wild type transcriptional activity of a baculovirus late and/or very late promoter, with the proviso that said modified factor still allows baculovirus replication.

In certain embodiments of this aspect of the present invention expression cassette C(-) of the kit further contains an open reading frame coding for the desired recombinant protein under the control of said promoter. In the latter case, the expression cassette becomes identical to the expression cassette C as defined herein.

The kits may further comprise instructions for using the components of the kit. Such instructions can be included in the kit in written, electronic, or any other suitable form, describing how to make and employ the recombinant baculovirus expression systems of the present invention.

Typically, baculovirus expression systems are derived from nuclear polyhedrosis viruses (NPV). While in principle all baculovirus expression systems can be modified to work in the context of the present invention, a preferred inducible baculovirus expression system is based on Autographa californica nuclear polyhedrosis virus (AcMNPV). Examples of other preferred viruses include any of the multiple nucleocapsids per envelope (MNPV) subgenera of the NPV genera of the Eubaculovirinae subfamily (occluded Baculoviruses) of the Baculoviridae family of insect viruses.

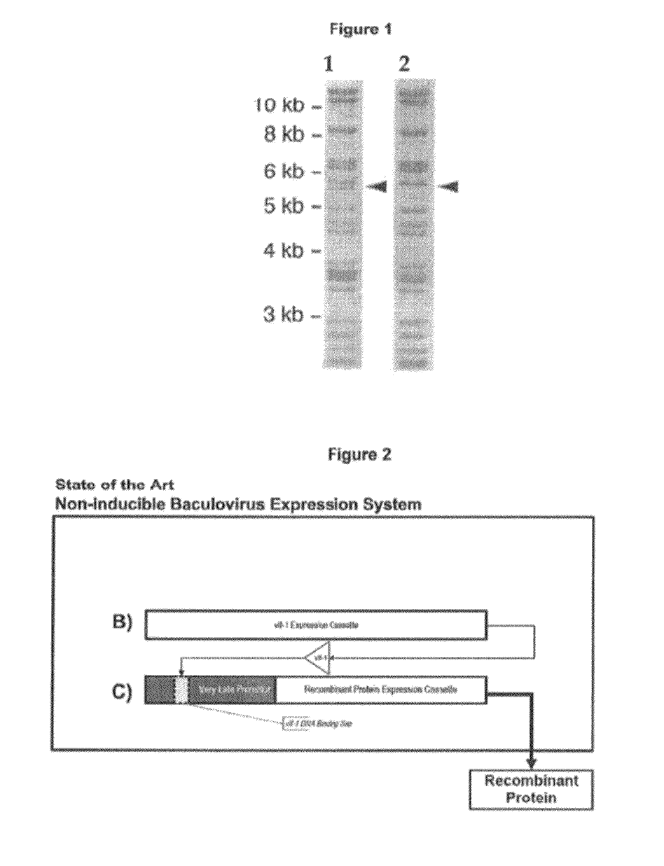

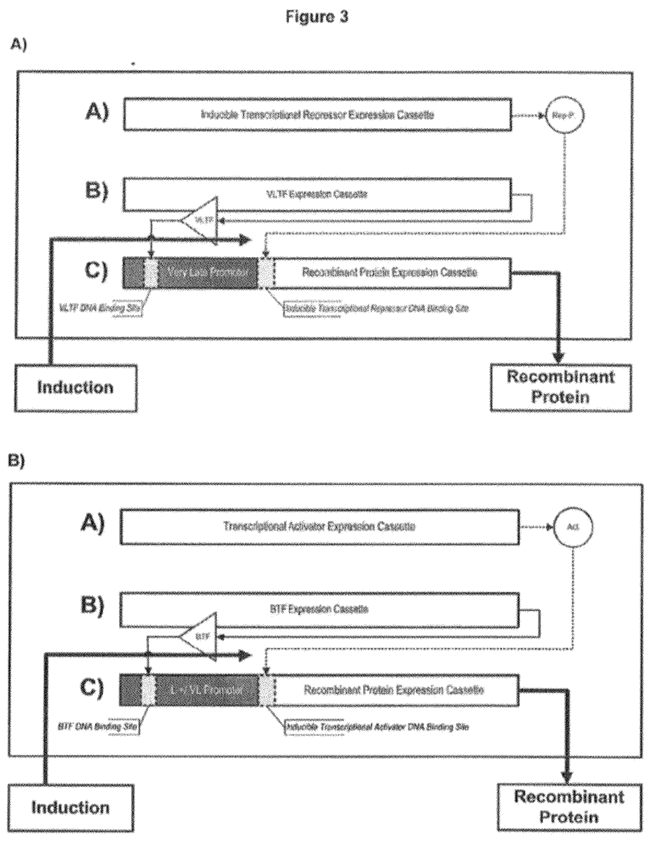

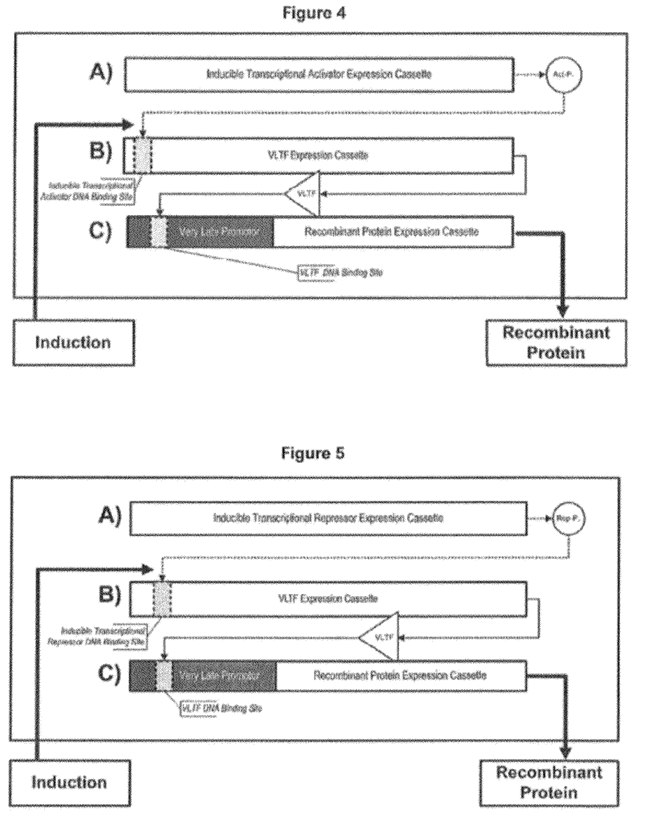

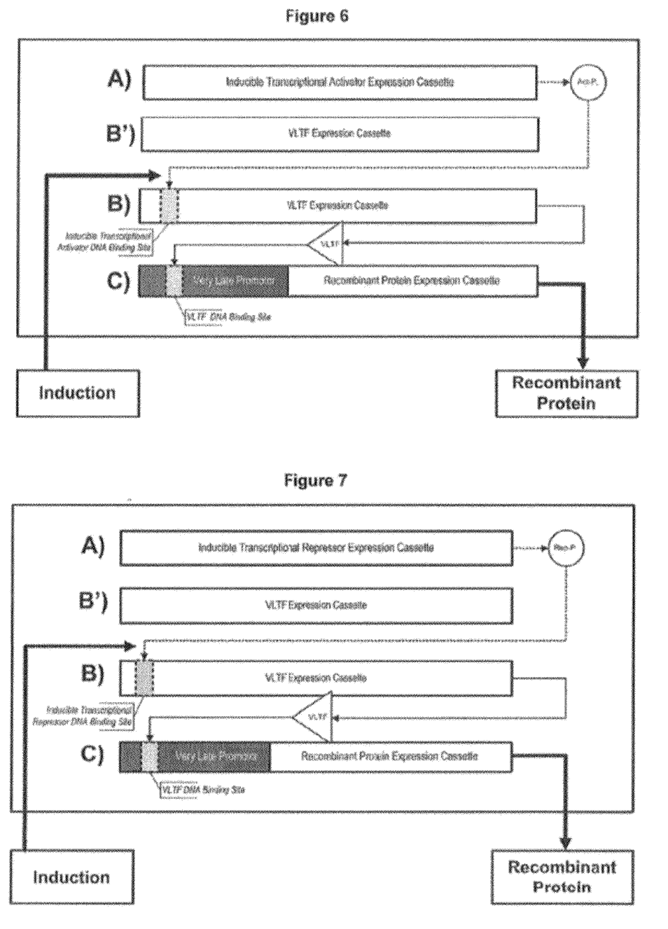

There are a number of commercial systems for expressing recombinant proteins using baculovirus, all based on AcMNPV. They include. flashBAC™ (Oxford Expression Technologies EP1144666), BackPack™ (BD Biosciences Clontech), BacVector® 1000/2000/3000 (Novagen®), BAC-TO-BAC® (Invitrogen™ U.S. Pat. No. 5,348,886), and BaculoDirect™ (Invitrogen™). All these baculovirus-based insect cell expression systems are essentially based on the architecture described in FIG. 2, expressing recombinant proteins by placing them under the control of very late baculovirus promoters, namely the polh and/or p10 promoters. Any of these baculovirus expression systems could be conveniently modified to comply with the present invention by incorporating controllable transcriptional activators or repressors into the system as described herein, thereby directly or indirectly controlling recombinant protein expression. Accordingly, the inducible expression systems of the present invention can be based on any one of the commercially or academically available baculovirus expression systems.

There are a vast number of well studied controllable transcriptional activators and repressors and their respective response elements available in the literature that can be used according to the invention. They come from prokaryotes, eukaryotes, or were created through molecular biological techniques. The majority of commercially available inducible systems have been engineered to improve or combine natural systems to suit the needs of a wide range of specialized scientists. For example, DNA binding domains from prokaryotes have been combined with activation domains from eukaryotes. Similarly, a wide array of synthetic chemical inducers or co-repressors have been described which function better than their natural structural analogues.

A controllable transcriptional modulator can be an inducer molecule controlled transcriptional activator protein, an inducer molecule (corepressor) controlled transcriptional repressor protein, a physically controlled transcriptional activator protein or a physically controlled transcriptional repressor protein.

Suitable non-limiting examples for inducer molecule-controlled transcriptional activators and their inducers are the protein metallothionein (MT) binding DNA sequences called metal responsive element (MREs) in the presence of its inducer, metals such as copper (CuSO₄, Nucl. Acids Res, 16, 1043-1061); AMT1, another metal responsive transcriptional activator (Proc. Natl. Acad. Sci. 88, 6112-6116); steroid-inducible transcriptional activators, glucocorticoid receptor (GC) proteins, binding GREs in the presence of steroid inducers such as cortisol or its structural analogue dexamethasone (Proc. Natl. Acad. Sci. 90, 5603-5607); estrogen receptor (ER) binding its DNA response elements under the influence of a wide range of molecules (Pharmacol. Rev. 58, 773-781); the alcohol-dependent transcription activator AlcR (Mol. Microbiol. 20, 475-488), the chimeric protein tTA and rtTA of the Tet-off and Tet-on system, respectively, binding to tetracycline response elements (TREs) controlled by tetracycline or derivatives such as doxycycline (Annu. Rev. Genet. 36, 153-73, PNAS 89, 5547-5551); the CAP protein and its inducer cAMP (JMB 293, 199-213); the AP-1 proteins and its inducer phorbol esters (Mol. Pharm. 56, 162-169); and WRKY1, WRKY2, and WRKY3 activating transcription in the presence of the oligopeptide elicitor Pep25 (EMBO J. 15, 5690-700).

Suitable but non-limiting examples for inducer molecule controlled transcriptional repressors and their inducers (corepressors) are the tetracyclin repressor protein TetR and its corepressor tetracycline or derivatives thereof such as doxycycline (EMBO 3, 539-43); the CymR repressor protein and its corepressor p-cumate (J. Bacteriol. 179, 3171-3180); the trpR repressor and its corepressor tryptophan (PNAS 79, 3120-3124); the PurR repressor and its corepressor guanine or hypoxanthine (Cell 83, 147-155), the MetJ repressor protein and its corepressor SAM (Proc. Natl. Acad. Sci. 106, 5065-5069); the lac repressor protein and its corepressor lactose and structural analogues thereof such as IPTG (C. R. Biol. 328, 521-48); and the tox repressor protein and its corepressor transition metals (PNAS 92, 6803-7). In particular embodiments of the invention, the presence of an inducer molecule stimulates expression of the recombinant protein that is under the control of the baculovirus late and/or very late promoter in expression cassette C.

Transcription can also be activated or repressed by environmental factors such as light or temperature. Non-limiting examples for physically-induced transcriptional activator proteins and their inducers are heat shock transcription factor (HSF) binding to heat shock promoter elements (HSE) with consensus nGAAnnTTCn, heat shock proteins (HSPs) such as the temperature responsive HSP system in Drosophila (Nature 327, 727-730; Nature 368, 342-344) with HSP70 and associated proteins such as Hap46 (Proc. Natl. Acad. Sci. 96, 10194-10199) or Drosophila HSP70 homologs such as Ssa 1 (Mol. Microbiology 62, 1090-1101). Other species have comparable systems such as the HSF1 transcriptional activator from Arabidopsis (Biol. Chem., 384, 959-963). Photocaged derivatives of hydroxytamoxifen and guanidine tamoxifen have been synthesized that selectively antagonize estrogen receptor (ER) activated transcription at classic estrogen response elements (ChemBioChem 5, 788-796).

Non-limiting examples for physically induced transcriptional repressor proteins are the bacterial hrcA repressor protein reversibly binding its CIRCE DNA element in response to changes in temperature (J. Bacteriol. 182, 14-22). Further, RheA is a temperature sensitive protein from Streptomyces albus (PNAS 97, 3538-3543) and HSF-4a is human temperature sensitive repressor protein (J. Cell. Biochem. 82, 692-703).

Preferably the controllable transcriptional repressor protein of the invention is selected from the group consisting of TetR, CymR, trpR, MetJ, lac repressor protein and tox repressor protein. The controllable transcriptional activator protein is preferably selected from the group consisting of metallothionein (MT), AMT1, Glucocorticoid receptor protein (GC), Estrogen receptor, AlcR, tetR-VP16, tTA, CAP, AP-1, WRKY1, WRKY2 and WRKY3.

In other embodiments of the invention, the transcriptional modulator protein, is not a hormone receptor, for example is one derived from a bacterial/prokaryotic DNA binding protein, such as one that is, or is derived from, a bacterial/prokaryotic transcriptional modulator protein.

In other embodiments of the present invention, the transcriptional repressor response element is not a hormone receptor response element, and in certain such embodiments the transcriptional modulator response element is derived from a bacterial/prokaryotic transcriptional modulator response element”.

As mentioned above, in preferred embodiments of the invention the factor is one known as “very late factor 1” (vlf-1) or a transcriptionally functional homolog thereof. The term a “transcriptionally functional homolog” thereof refers to a protein that shows a comparable transcriptional activity as vlf-1 of Seq ID No: 3 or encoded by Seq ID No: 4 or Seq ID No. 10, wherein comparable means at least 80%, preferably 85%, 90%, 95%, 100% or >100% of the transcriptional activity of “wild type” vlf-1 of Seq ID No: 3. Methods for determining transcriptional activity of a protein are known in the art and can be determined for example by methods described herein. Even more preferably the transcriptionally functional homolog of vlf-1 further is at least 70% identical to vlf-1 of Seq ID No: 3 on the amino acid level, more preferably at least 75%, 80%, 85%, 90%, 95%, 98% or 99% identical to vlf-1 on the amino acid level. According to the invention transcriptionally functional homologs of vlf-1 also include fragments of vlf-1 protein with the transcriptional activity being at least 80%, preferably 85%, 90%, 95%, 100% or >100% of the transcriptional activity of vlf-1 of Seq ID No: 3, determined as described above.

In certain embodiments the vlf-1 gene is expressed under the control of a promoter, wherein promoter means “original” or a functional homolog thereof. The “original” promoter of vlf-1 from AcMNPV is given in Seq ID No: 5 and is one preferred embodiment of the present invention.

Alternatively vlf-1 is expressed under the control of a weak promoter. One example of a weak promoter for expression of vlf-1 is the vhspRVvlf1-derived promoter of Seq ID No: 9 (Virology 248, 131-138), but any promoter transcribing lower levels of vlf-1 than the “original” promoter of Seq ID No: 5 is a weak promoter according to the invention. As known in the art promoters can for example be weakened by inserting a DNA sequence between the transcription and translation start.

In certain preferred embodiments the modified factor (such as modified VLTF) is modified vlf-1, wherein “modified vlf-1” as used herein refer to a vlf-1 protein allowing baculovirus replication but having markedly decreased very late promoter transcriptional activity. Examples of modified vlf-1 proteins in accordance with the invention are vlf-1 of Seq ID No: 3 (or of NCBI accession number NP_{—}054107) carrying a C202Y mutation (J. Virology 68, 7746-7756) or a cysteine insertion between P23 and R24 (vcBsuSsevlf1, Virology 248, 131-138). Both mutants allow baculovirus replication while showing a markedly decreased very late promoter transcriptional activity. Other suitable modifications would be any modification affecting binding to very late promoters without blocking viral replication. Since lower levels of vlf-1 are required for supporting replication than for the transcription of the genes under the control of the very late promoter any mutation reducing vlf-1 protein or mRNA stability would appear to be suitable in the context of this embodiment of the present invention.

In another aspect, the present invention relates to a recombinant nucleic acid, such as one comprising expression cassette C(-) as defined or otherwise described herein. For example, such a recombinant nucleic acid can comprise at least: (i) the promoter of expression cassette C of the inducible baculovirus expression system of the present invention; (ii) a transcriptional modulator response element; and (iii) a cloning site, such as one for inserting, a nucleic acid. Suitable cloning/insertion sites for the recombinant nucleic acids of the invention will be readily known to the person of ordinary skill and include (multiple) cloning sites that can be digested by one or more restriction enzyme, and/or recombination-based insertion sites such as those used for the “Gateway” cloning system of Invitrogen of that use Cre-Lox system.

In certain embodiments of the recombinant nucleic acids of the invention, the transcription of a nucleic acid inserted into the cloning site is under the control of the promoter therein, and such transcription may be modulated by a controllable transcriptional modulator protein reversibly interacting, in one condition, with the transcriptional modulator response element of said recombinant nucleic acid. In other embodiments, said transcriptional modulator response element is in, or in proximity (adjacent) to, said promoter.

In particular embodiments of the recombinant nucleic acids of the invention, said transcriptional modulator response element is not a hormone receptor response element. In other embodiments, said transcriptional modulator response element binds to a bacterial/prokaryotic controllable transcriptional modulator protein and/or is (or is derived from) a bacterial/prokaryotic transcriptional modulator response element.

In a particular embodiment of the recombinant nucleic acids of the invention, said transcriptional modulator response element may bind to, may be capable of binding, be controlled by or may be controlled by a controllable transcriptional repressor protein.

In other certain embodiments, the recombinant nucleic acids of the invention may further comprise an open reading frame for a recombinant protein inserted within said cloning site.

In another aspect, the present invention relates to a vector, such as a bacmid. comprising a recombinant nucleic acid of the invention. In certain embodiments, the vector of the invention is useful for fusion with baculovirus DNA, including with modified baculovirus DNA, and in particular such embodiments the vector is a baculovirus transfer vector. As will be appreciated by the person of ordinary skill, such vector will also comprise other features that assist the maintenance and/or replication of the vector in a cell, such as in a cell described herein.

In yet another aspect, the present invention relates to a composition that includes a recombinant nucleic acid of the invention. A composition of the present invention includes any mixture of two or more components one of which includes a recombinant nucleic acid of the invention as defined, claimed or otherwise described herein. In certain embodiments of such aspect, the composition is a two component mixture including such nucleic acid and at least one other component useful for the construction, and/or practice of the methods using, the inducible baculovirus expression system of the invention. Such other component may include a nucleic acid encoding expression cassette A and/or expression cassette B. Alternatively, such other component(s) may comprise the controllable transcriptional modulator protein, such as the controllable transcriptional repressor protein that reversible interacts with the transcriptional modulator response element of said nucleic acid. In other embodiments of this aspect of the invention, the composition may be a complex mixture, such as that of, or otherwise found in, a cell-free transcription/translation system, a cell-extract or an intact cellular environment. In particular such embodiments, the inventive composition that includes a recombinant nucleic acid of the invention is a cell, such as a bacterial, yeast, insect or mammalian cell, for example such a cell comprised in in-vitro or industrial tissue culture or storage.

In certain embodiments, the recombinant nucleic acid, vector or composition of the invention is useful for repressing expression of a recombinant protein encoded by an open reading frame inserted into said cloning site, for example when amplifying baculovirus in insect cells

In a further aspect, the present invention relates to modified baculovirus DNA that comprises expression cassette B of the inducible baculovirus expression system of the invention, wherein a) the expression cassette B contains an open reading frame coding for said factor under the control of a weak promoter and further comprises a transcriptional activator response element; or b) the expression cassette B comprises a transcriptional repressor response element.

In particular embodiments, the recombinant nucleic acid, the vector, the composition or the modified baculovirus DNA of the invention, further comprises expression cassette A of the inducible baculovirus expression system of the invention.

In yet a further aspect, the present invention relates to a method of repressing production of a recombinant protein in an insect cell, said method comprising: a) providing insect cells of the invention; and b) maintaining said insect cells under conditions wherein the expression of the recombinant protein encoded by expression cassette C of said inducible baculovirus expression system is (directly or indirectly) repressed by the controllable transcriptional modulator protein encoded by expression cassette A of said inducible baculovirus expression system.

In a related aspect the invention also relates to a method of repressing transcription of an open reading frame coding for a recombinant protein during baculovirus amplification in insect cells, said method comprising: a) providing insect cells of the invention; and b) maintaining said insect cells under conditions wherein the transcription of the open reading frame contained in expression cassette C of said inducible baculovirus expression system is (directly or indirectly) repressed by the controllable transcriptional modulator protein encoded by expression cassette A of said inducible baculovirus expression system.

In certain embodiments of the various methods of the invention, said method includes conditions that allow replication (and/or amplification) of baculovirus in insect cells.

Another aspect of the present invention relates to a method of increasing the culture volume of recombinant insect cells, said method comprising: a) introducing the inducible baculovirus expression system of the invention or the composite baculovirus DNA of the invention into a first culture of insect cells; b) culturing said insect cells under conditions that allows replication of baculovirus; and c) introducing baculovirus obtained from step b) into a second culture of insect cells, wherein said second culture of insect cells has a larger total volume than the first culture of insect cells; and further wherein transcription of the open reading frame contained in expression cassette C of said inducible baculovirus expression system is repressed by the controllable transcriptional modulator protein encoded by expression cassette A of said inducible baculovirus expression system. In certain of such embodiments of this method, the second culture of insect cells is about 2, 4, 5, 10, 50, 100, 250, 500, 1,000. 5,000 or 10,000 times larger than said first culture, and in particular embodiments is greater than 10,000 larger than said first culture such as about 100,000 or 1,000,000 times larger. In other certain embodiments of this method, the steps of the method are repeated, preferably are repeated between 2 and about 10 times, such as repeated 3, 4, 5, 6, 7 or 8 times.

A further aspect of the present invention relates to a method of reducing the rate at which recombinant protein expression cassettes are mutated and/or eliminated from recombinant baculovirus being amplified in insect cells, said method comprising: a) amplifying in insect cells the inducible baculovirus expression system of the invention or the composite baculovirus DNA of the invention; and b) controlling the controllable transcriptional modulator protein encoded by expression cassette A of said inducible baculovirus expression system under conditions that repress the transcription of the open reading frame contained in expression cassette C of said inducible baculovirus expression system.

Yet one further aspect of the present invention relates to a method of amplifying baculovirus in insect cells, said method comprising: a) providing insect cells of the invention; and b) maintaining said insect cells under conditions that allow replication of baculovirus; wherein transcription of the open reading frame contained in expression cassette C of said inducible baculovirus expression system is repressed, during said replication, by the controllable transcriptional modulator protein encoded by expression cassette A of said inducible baculovirus expression system.

In certain embodiments of any of the various methods of the present invention, said repression is brought about by the controllable transcriptional modulator protein (encoded by expression cassette A of said inducible baculovirus expression system) reversibly interacting with the transcriptional modulator response element (of said inducible baculovirus expression system) in one condition, and (reacting/interacting) differently in a second condition, thereby modulating the transcription of expression cassette B or expression cassette C of said inducible baculovirus expression system. In particular such embodiments, said repression is, is effected by or is otherwise directly or indirectly caused by, a transcriptional repressor protein binding to a transcriptional repressor response element in expression cassette B or expression cassette C.

In other particular embodiments of the methods of the invention, interaction of the controllable transcriptional modulator protein to its transcriptional modulator response element causes (or effects) said repression, preferably wherein said transcriptional modulator response element is in expression cassette C of said inducible baculovirus expression system.

In other certain embodiments of the methods the controllable transcriptional modulator protein is one that can be controlled by an inducer molecule, and said repression comprises the presence of, or in alternative embodiments the absence of, said inducer molecule.

In those methods of the present inventions defined, claimed or otherwise described herein that are not directed to the production of a recombinant protein, certain embodiments of such methods further comprise the step of inducing transcription of the open reading frame in expression cassette C that encodes a recombinant protein.

In particular embodiments of the methods of the invention that comprise the induction of expression of a recombinant protein, said induction comprises the presence of an inducer molecule with the inducible baculovirus expression system, preferably by addition of the inducer molecule to said culture or maintenance conditions.

As will now be apparent to the person of ordinary skill, the various methods of the present invention have particular advantages in the context of large scale (such as industrial) production of recombinant protein. Accordingly, in certain embodiments of the various methods of the invention, the method includes a step of culturing insect cells that comprises conditions under which baculovirus is amplified in said insect cells, under which the number of insect cells increases and/or under which the number of baculovirus particles is increased. In those methods including a step comprising culture conditions for baculovirus amplification, said conditions comprise between 2 and about 10 rounds of virus amplification, such as comprising 3, 4, 5, 6 or 8 rounds of virus amplification.

In certain embodiments of the method claims, the culture conditions are maintained (and/or repeated), or the various steps of the method are repeated until the number of insect cells is between about 10⁸ and 10³, such as between about 10⁹ and 10¹² cells, and/or until the number of baculovirus particles is between 10 ⁸ and 10¹³, such as between about 10⁹ and 10¹² virus particles. In alternative embodiments, said conditions are maintained until the total volume of culture is between about 0.1 L and 10,000 L. such as between about 100 and 1,000 L, and/or are maintained for a period of time that is between about 1 day and 3 weeks, such as between about 3 days and 1 week after the introduction of the inducible baculovirus system of the invention into the insect cell or the provision (and/or start of culture) of the insect cell comprising such a system.

In particular embodiments of the various aspects of the present invention, expression cassette A further comprises a (second) transcriptional modulator response element, preferable in or in proximity (adjacent) to the promoter of expression cassette A, wherein said (second) transcriptional modulator response element reversible interacts with a controllable transcriptional modulator protein in one condition, and (reacts/interacts) differently in a second condition, thereby modulating the transcription of expression cassette A. Without being bound by theory, the inclusion of a transcriptional modulator response element in expression cassette A, and its use to control (over) expression of the controllable transcriptional modulator protein itself, is thought to further reduce selective pressure on the inducible baculovirus expression system of the present invention, enabling virus to be amplified yet more effectively (see Example 6). In certain of such embodiments, the controllable transcriptional modulator protein is a controllable transcriptional repressor protein. In other certain of such embodiments, said (second) transcriptional modulator response element expression cassette A comprises the same sequence as the (first) transcriptional modulator response element in expression cassette C, and can bind to the same controllable transcriptional modulator protein. In yet other such embodiments, the controllable transcriptional repressor protein can auto-repress its transcription.

Other aspects of the present invention relate to a kit, such as a kit of parts, that includes a plurality of components for the construction and/or use of an inducible baculovirus expression system of the present invention. Such plurality of components may be presented, packaged or stored separately. For example, they may be isolated from one another by being held in separate containers. Accordingly, one embodiment of such a kit of the invention comprises at least two components that include (preferably separately): (i) the recombinant nucleic acid, vector, composition or the modified baculovirus DNA of the invention; and (ii) at least one other component for the construction and/or use of an inducible baculovirus expression system. Such components, although held separately, may be boxed or otherwise associated together to aid storage and/or transport, and such association may include additional components. In particular embodiments of such kits of the invention, at least one of the second (or additional) components may comprise: (i) the expression cassette A of the inducible baculovirus expression system of any of the claims above, or a vector or a cell that comprises said expression cassette; (ii) an insect cell, preferably one selected from the selected from the group consisting of insect cells derived from Spodoptera frugiperda, Trichoplusia ni, Plutella xylostella, Manduca sexta, and Mamestra brassicae, such as an insect cell that is a IPLB-SF21AE cell or its clonal isolate Sf9; (iii) an inducer molecule that modulates the reversible interaction of a controllable transcriptional modulator protein with a transcriptional modulator response element; and/or (iv) instructions describing how to construct and/or use the inducible baculovirus expression system of the present invention, and/or to practice any of the various methods of the present invention.

It will be appreciated that the following examples are intended to illustrate but not to limit the present invention. Various other examples and modifications of the foregoing description and examples will be apparent to a person skilled in the art after reading the disclosure without departing from the spirit and scope of the invention. In fact, those of ordinary skill in the art will appreciate that baculovirus systems and components thereof and methods other than those specifically described herein can readily be employed in the practice of this invention as broadly described herein without undue experimentation. All baculovirus systems and components thereof and methods that can be readily adapted to the practice of this invention or that are recognized in the art to be functionally equivalents of the specific baculovirus systems and components thereof and methods disclosed herein are intended to be encompassed by the appended claims.

Moreover, all references cited are incorporated by reference in their entirety to the extent that they are not inconsistent with the description herein.

### EXAMPLES

### Example 1

FIG. 1 illustrates the phenomenon of specific loss of recombinant protein expression cassettes during scale-up using baculovirus. Here, purified baculovirus DNA was analyzed by agarose gel electrophoresis to examine what occurs to recombinant protein DNA expression cassettes before (lane 1) and after (lane 2) scale-up. In FIG. 1, lane 2 the DNA band corresponding to the recombinant protein expression cassette from lane 1 almost completely disappeared during virus passaging and its disappearance was correlated with an almost complete loss of recombinant protein production (not shown). Presumably the loss of the recombinant protein DNA expression cassette seen in lane 2 is due to the fact that the extra metabolic cost associated with both transcribing and translating very heavily expressed, non-essential recombinant genes during viral amplification led to selective pressure to specifically eliminate the recombinant protein DNA expression cassette.

### Example 2

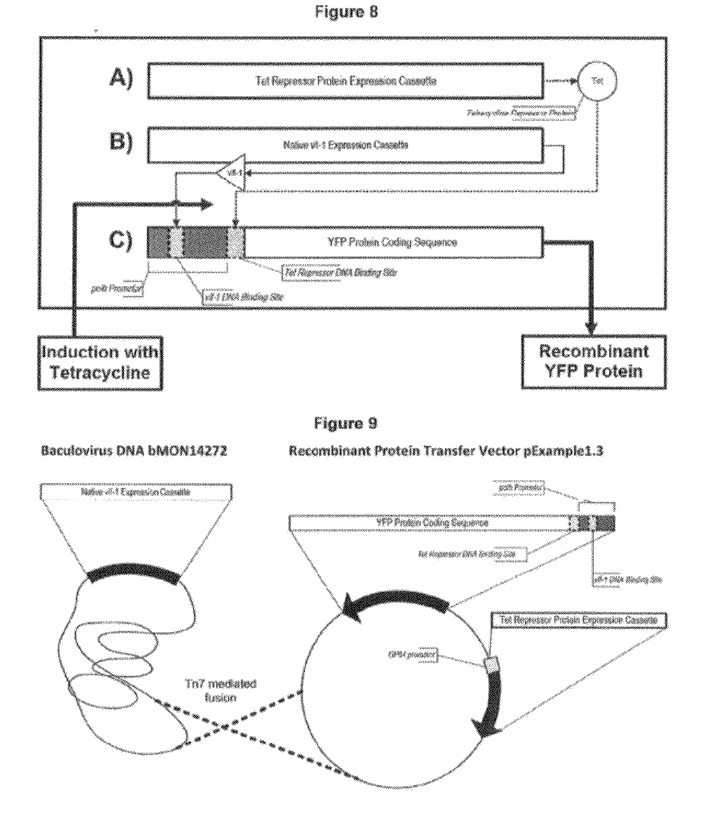

As shown in FIG. 8 an inducible transcriptional repressor protein, the tet repressor, is the product of expression cassette A. The product of expression cassette B is the native vlf-1 protein. The tet repressor protein has a binding site just downstream of the polh promoter in expression cassette C, while vlf-1 protein binds DNA within the polh promoter in expression cassette C. In the absence of tetracycline, the tet repressor protein remains bound to its DNA binding site in C and thereby inhibits vlf-1 activated transcription of C. When tetracycline is provided to the system, transcription, and thereafter translation of C is induced. The protein product of C is the fluorescent protein EYFP. EYFP production by baculovirus-infected insect cells in the presence and absence of tetracycline is quantified by measuring absorbance of EYFP.

The strategy for creation of composite baculovirus containing of the expression cassettes A, B, and C is described schematically in FIG. 9. Here B is the native vlf-1 protein located on the baculovirus DNA bMON14272 (J. Virology 67, 4566-4579). Cassettes A, coding for the tet repressor protein, and C, coding for EYFP, are located on a transfer vector called pEXAMPLE1.3. Composite baculovirus DNA containing expression cassettes A, B and C is created by fusing transfer vector pEXAMPLE1.3 with bMON14272. This is carried out by transforming pEXAMPLE1.3 into DH10BAC (Invitrogen) E. coli cells harboring the bacmid bMON14272 and the helper plasmid pMON712417. Tn7 mediated recombination in the DH10BAC cells then mediates the fusion of bMON14272 with pEXAMPLE1.3 as described in (J. Virology 67, 4566-4579). The same strategy is used to fuse pEXAMPLE1.2 (see below) with bMON14272.

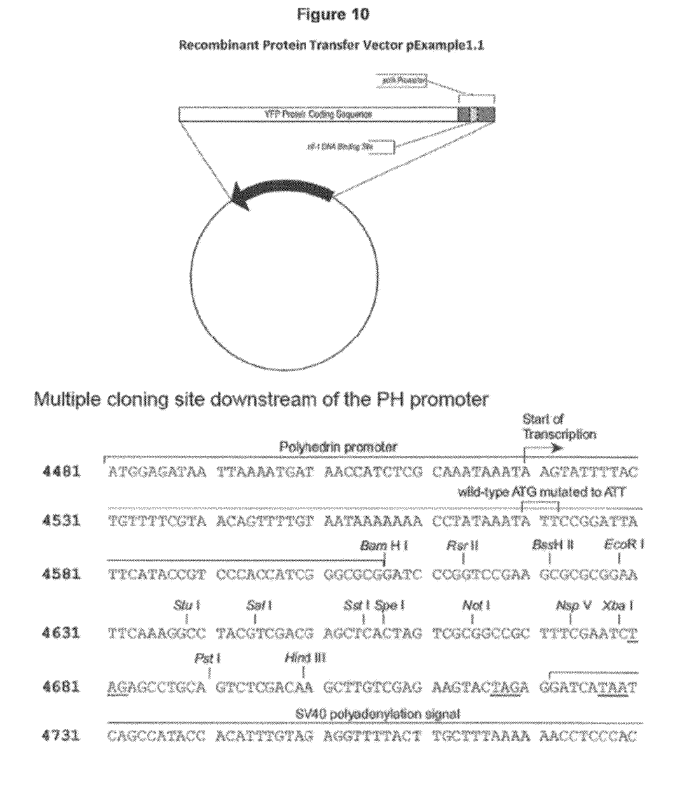

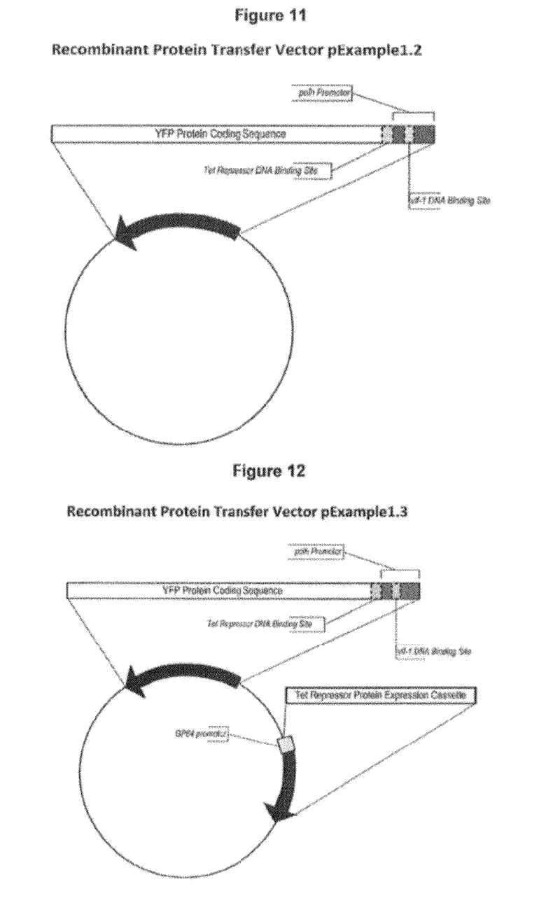

Transfer vector pEXAMPLE1.3 containing expression cassettes for EYFP and tet repressor protein was constructed starting with plasmid pFBDM from (Nature Biotechnology 22, 1583-1587). First, to construct pEXAMPLE1.1 (see FIG. 10), the EYFP coding sequence is placed under the polh very late promoter by subcloning a BamHI AwII fragment from plasmid pUCDM-EYFP (Nature Biotechnology 22, 1583-1587) into the BamHI AwII site of pFBDM. Next, complimentary annealed oligonucleotides comprising two tandem copies of the tetO1 sequence having Seq ID No: 6 (5′-ACTCTATCATTGATAGAGT-3′ from J. Mol. Biol. 202, 407-415) are cloned into the BamHI site of pEXAMPLE1.1 to yield pEXAMPLE1.2 (FIG. 11). To create pEXAMPLE1.3 (FIG. 12), an expression cassette containing the Tn10 tet repressor protein coding sequence having Seq ID No: 7 (EMBO 3, 539-43) under the control of the GP64 promoter from AcMNPV (NCBI accession number NC_{—}001623) having Seq ID No: 8 is created by gene synthesis and subcloned into BstZ17I and KpnI digested pEXAMPLE1.2.

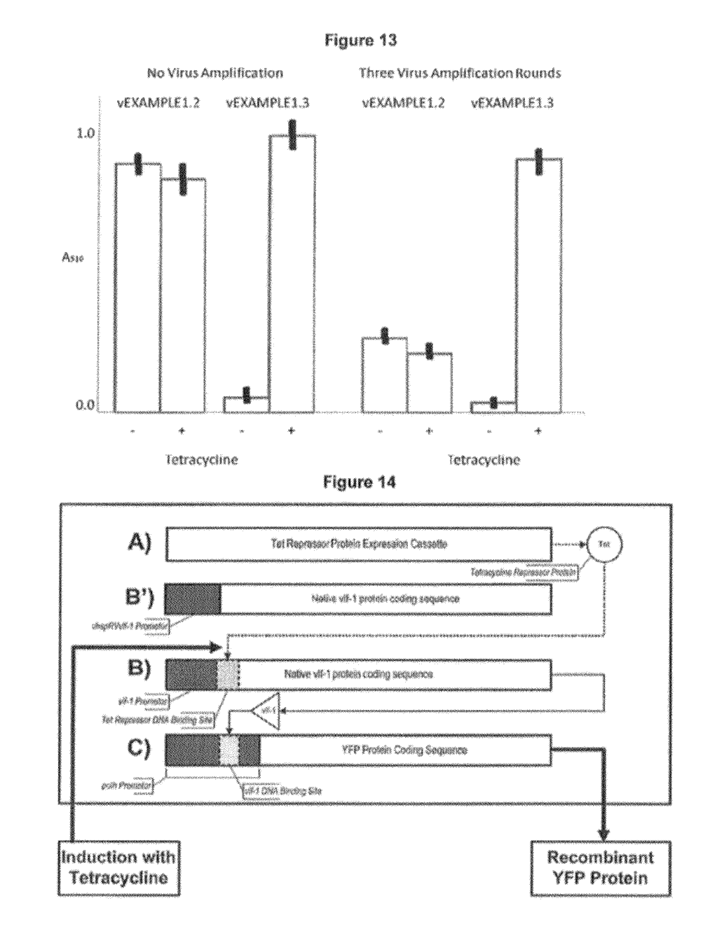

To create composite baculovirus bMON14272-pEXAMPLE1.2 and baculovirus bMON14272-pEXAMPLE1.3, composite bacmid DNA is isolated from DH10BAC following Tn7 transposition and transfected into SF21 insect cells according to (Nature Methods 3, 1021-1032). bMON14272-pEXAMPLE1.2 and bMON14272-pEXAMPLE1.3 initial transfection virus and infected cells are then harvested and used for subsequent expression tests (FIG. 13, no virus amplification). Initial transfection virus bMON14272-pEXAMPLE1.2 and bMON14272-pEXAMPLE1.3 is serially passaged at multiplicity of infection (M.O.I) 0.1 for two further generations with virus harvested at 48 hours post infection and also used for subsequent expression tests (FIG. 13, three virus amplification rounds).

Expression tests are carried out as follows. SF21 cells seeded at 70% confluency in a 6-well plate are infected at M.O.I 1.0 according to (Nature Methods 3, 1021-1032) either in the presence, or absence of 5 μg/ml tetracycline. Following 72 hours incubation, cells are lysed in 50 mM Tris, 50 mM NaCl, 0.1% NP40 pH 7.2, and absorbance at 510 nm is measured on a Beckman DU 640 spectrophotometer. Cells from bMON14272-pEXAMPLE1.3+tetracycline are diluted to reach an absorption at 510 nm (A₅₁₀) of 1.0 and a comparable number of cells from the other conditions outlined above are compared for A₅₁₀. As can be seen from FIG. 13, expression of EYFP is greater in the inducible system both before virus amplification and after three virus amplification rounds.

### Example 3

As shown in FIG. 14 an inducible transcriptional repressor protein, the tet repressor, is the product of expression cassette A. Component B comprises two expression cassettes, both coding for the native vlf-1 protein. One expression cassette, called B′, contains vlf-1 under control of the vhspRVvlf1-derived virus promoter described in (Virology 248, 131-138). This promoter produces low levels of vlf-1 which is sufficient to support normal viral replication without significantly stimulating polh transcription (Virology 248, 131-138). The second vlf-1 expression cassette called B, codes for the native vlf-1 protein and is driven by its native promoter but has a DNA binding site for the tet repressor between its promoter sequence and start codon. Vlf-1 proteins produced by B′ and B bind to their native DNA binding site within the polh promoter in expression cassette C. In the absence of tetracycline, the tet repressor protein remains bound to its DNA binding site in B inhibiting transcription and resulting in low levels of available vlf-1 protein. When tetracycline is provided to the system to release the tet repressor from its DNA binding site, transcription of B increases, increasing levels of available vlf-1 protein. This results in an increase in polh driven transcription of C. The protein product of C is the fluorescent protein EYFP. EYFP production by baculovirus infected insect cells in the presence and absence of tetracycline is quantified by measuring absorbance of EYFP.

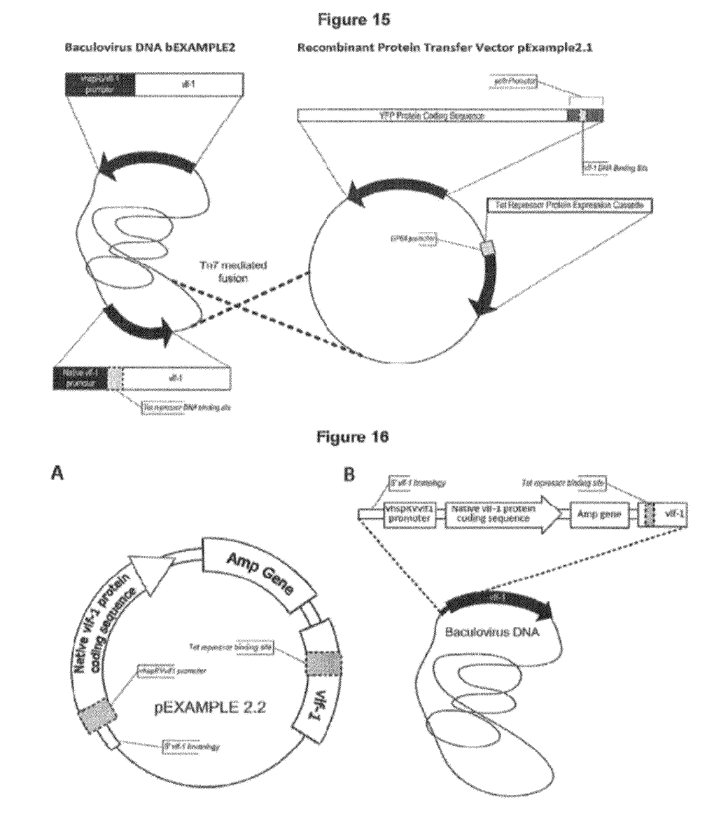

The strategy for making a composite baculovirus containing expression cassettes A, B′, B, and C is summarized in FIG. 15. Here the two vlf-1 expression cassettes B′ and B are both located on the baculovirus DNA bEXAMPLE2 which is a derivative of bMON14272. Cassettes A, coding for the tet repressor protein, and C, coding for EYFP, are located on a transfer vector called pEXAMPLE2.1. Composite baculovirus DNA containing expression cassettes A, B′, B and C is created by fusing transfer vector pEXAMPLE2.1 with bEXAMPLE2. This is carried out by transforming pEXAMPLE2.1 into competent E. coli cells containing bEXAMPLE2 and the helper plasmid pMON712417 (J. Virology 67, 4566-4579). Tn7 mediated recombination in the E. coli cells then mediates the fusion of pEXAMPLE2.1 with bEXAMPLE2 as described in (J. Virology 67, 4566-4579).

Recombinant protein transfer vector pEXAMPLE2.1 containing expression cassettes for EYFP and tet repressor protein is constructed in parallel to pEXAMPLE1.3 and is identical to pEXAMPLE1.3 with the exception that the tetO sequence is not subcloned into pEXAMPLE2.1.

To introduce two modified vlf-1 expression cassettes into the baculovirus genome, plasmid pEXAMPLE2.2 is created by gene synthesis (see FIG. 16 below). In pEXAMPLE2.2 vlf-1 under control of the vhspRVvlf1-derived weak promoter of Seq ID No: 9 flanks an amp marker on the 5′ side while native vlf-1 containing a tet repressor binding site is 3′ of the amp marker. The coding region of vlf-1 under control of the weak vhspRVvlf1-derived promoter (cassette B′) is created using a codon optimized Spodoptera frugiperda DNA sequence (www.kazusa.or.ip/codon/S.html) of Seq ID No: 10 in order to avoid direct DNA repeats between expression cassette B and expression cassette B′.

To introduce the two modified copies of vlf-1 into the baculovirus genome, baculovirus DNA is modified through ET recombination (Nat. Genet. 20, 123-128) using the protocols described in (Nature Biotechnology 22, 1583-1587).

Briefly, the vector pBAD-ETgamma carrying truncated recE under the arabinose-inducible PBAD promoter and recT under the EM7 promoter is modified by placing the zeocin resistance gene from pPICZA into the FspI and ScaI sites as described for pBADZ-His6Cre yielding pBADZ-ETgamma. This vector is transformed into DH10BAC (Invitrogen) cells harboring the bacmid bMON14272 and the helper plasmid pMON712417 and a kanamycin, tetracyclin and zeocin resistant colony is cultured and I-arabinose added to 0.1% and the culture is incubated until OD600=0.5. Electro-competent DH10BACET cells are then generated following standard procedures and stored at −80° C.

Next, to integrate the desired 2 copies of vlf-1 in the baculovirus DNA to create bEXAMPLE2, 5 μg of a phosphorylated PCR product from pEXAMPLE2.2 (see FIG. 16B) is electroporated into DH10BACET cells. Transformed cells are grown for 4 h at 37° C. and plated on agar plates containing kanamycin, tetracycline and ampicillin. Bacmid DNA from two single triple-resistant colonies is analyzed by PCR to confirm correct integration. Integrants are made electro-competent according to (Nature Biotechnology 22, 1583-1587) and recombinant protein transfer vector pEXAMPLE2.1 is introduced into this modified baculovirus DNA in the same manner as for pEXAMPLE1.3 described above.

To create composite baculovirus bEXAMPLE2-pEXAMPLE2.1 composite bacmid DNA is isolated from bEXAMPLE2 following Tn7 transposition of pEXAMPLE2.1 and transfected into SF21 insect cells according to (Nature Methods 3, 1021-1032). bMON14272-pEXAMPLE1.2 and bEXAMPLE-pEXAMPLE2.1 initial transfection virus is then harvested and used for subsequent expression tests (FIG. 17, no virus amplification). In addition, initial transfection virus bMON14272-pEXAMPLE1.2 and bMON14272-pEXAMPLE2.1 is serially passaged at multiplicity of infection (M.O.I) 0.1 for two further generations with virus harvested at 48 hours post infection and is also used for subsequent expression tests (FIG. 17, three virus amplification rounds).

Expression tests are carried out as follows. SF21 cells seeded at 70% confluency in a 6-well plate are infected at M.O.I 1.0 according to (Nature Methods 3, 1021-1032) either in the presence, or absence of 5 μg/ml tetracycline. Following 72 hours incubation, cells are lysed in 50 mM Tris, 50mM NaCl, 0.1% NP40 pH 7.2, and absorbance at 510 nm is measured on a Beckman DU 640 spectrophotometer. Cells from bEXAMPLE2-pEXAMPLE2.1+tetracycline from are diluted to reach A510 of 1.0 and a comparable number of cells from the other conditions outlined in FIG. 11 are compared for A₅₁₀. As can be seen from FIG. 17, expression of EYFP is greater in the inducible system both after no virus amplification and after three virus amplification rounds.

### Example 4

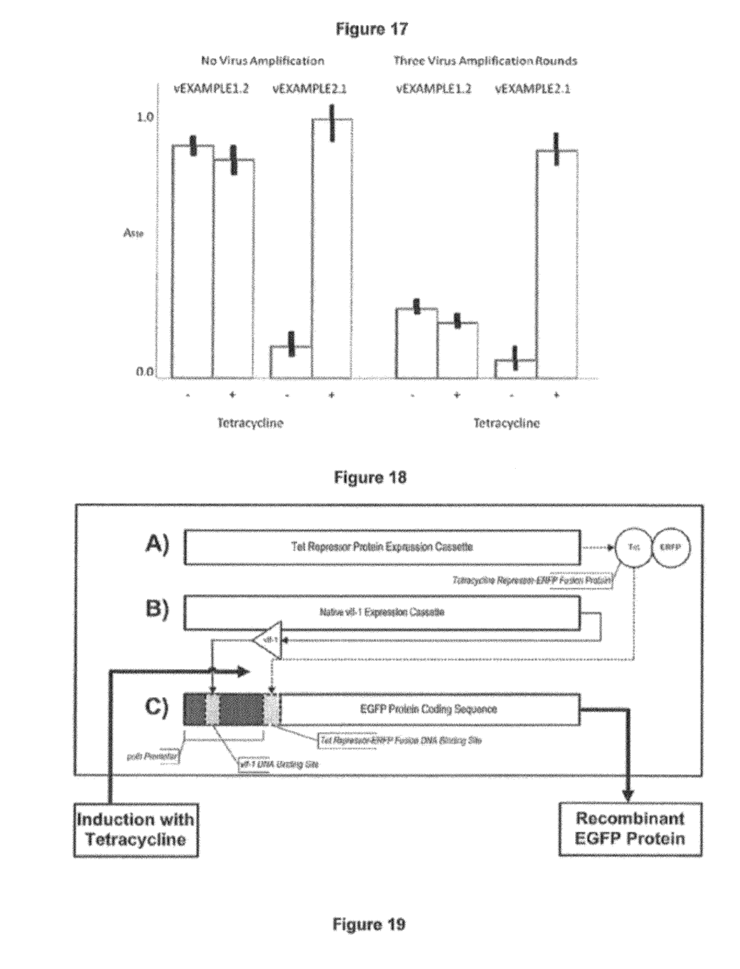

As shown in FIG. 18, an inducible transcriptional repressor protein, the tet repressor protein, fused to the fluorescent protein ERFP for detection purposes, is the product of expression cassette A. The product of expression cassette B is the native vlf-1 protein. The tet-ERFP fusion protein has a binding site just downstream of the polh promoter in expression cassette C, while vlf-1 protein binds DNA within the polh promoter in C. In the absence of tetracycline, the tet-ERFP fusion protein (controllable transcription modulator protein) remains bound to its DNA binding site (transcriptional modulator response sequence) in C and thereby inhibits vlf-1 activated transcription of C. When tetracycline is provided to the system, the tet-ERFP fusion dissociates from its binding site in C and thereby expression of C is induced. The protein product of C is the fluorescent protein EGFP. EGFP and ERFP were chosen to monitor two separate protein expression cassettes because they do not display significant overlap in their respective absorption spectrums. EGFP production by baculovirus-infected insect cells in the presence and absence of tetracycline is quantified by measuring absorbance of EGFP, and by western blot detection of EGFP.

The strategy for creation of composite baculovirus containing of the expression cassettes A, B, and C is described schematically in FIG. 19. Here B, the native vlf-1 protein is located on the baculovirus DNA bMON14272 (J. Virology 67, 4566-4579). Cassettes A, coding for the tet-ERFP fusion protein, and C, coding for EGFP, are located on a transfer vector called pEXAMPLE3.3. Composite baculovirus DNA containing expression cassettes A, B, and C, was created by fusing transfer vector pEXAMPLE3.3 with bMON14272. This was carried out by transforming pEXAMPLE3.3 into DH10 BAC (Invitrogen) E. coli cells harboring the bacmid bMON14272 and the helper plasmid pMON712417. Tn7 mediated recombination in the DH10BAC cells then mediated the fusion of bMON14272 with pEXAMPLE3.3 as described in (J. Virology 67, 4566-4579).

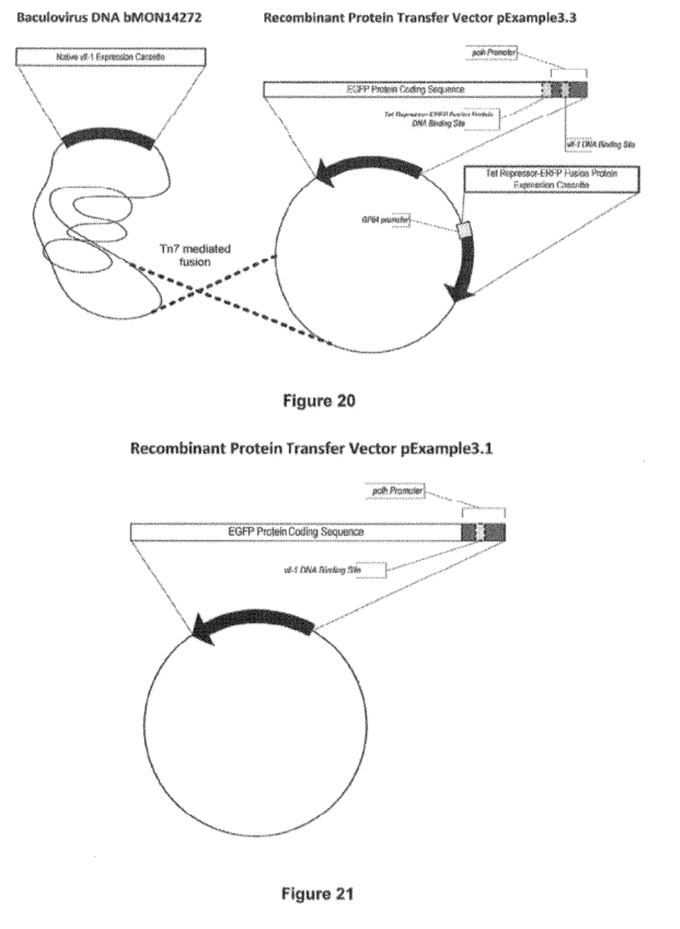

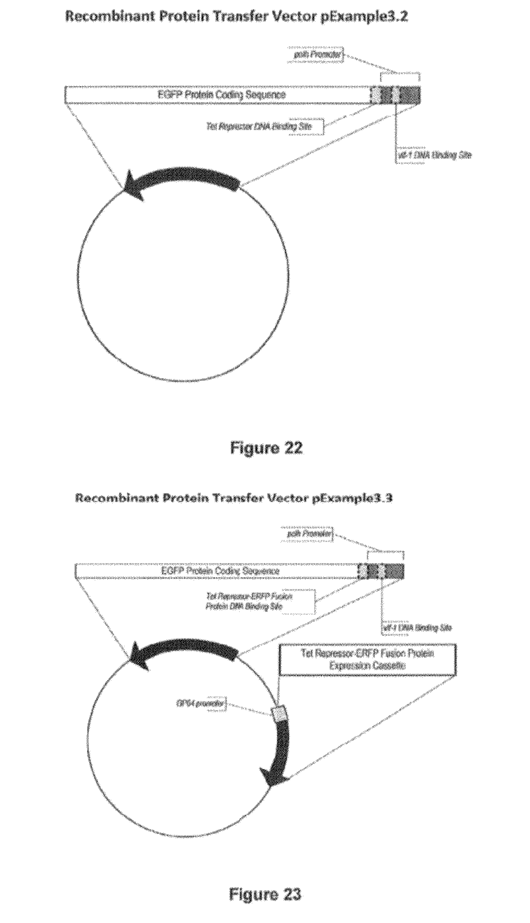

Transfer vector pEXAMPLE3.3 containing expression cassettes for EGFP and tet repressor-ERFP fusion protein was constructed starting with plasmid pFBDM from (Nature Biotechnology 22, 1583-1587). First, to construct pEXAMPLE3.1 (see FIG. 20), the EGFP coding sequence was placed under the polh very late promoter by subcloning a BamHI-XbaI EGFP fragment from pEGFP (Clontech) into the BamHI-XbaI site of pFBDM. Next, complimentary annealed oligonucleotides comprising two tandem copies of the tetO1 sequence having Seq ID No: 6 (5′-ACTCTATCATTGATAGAGT-3′ from J. Mol. Biol. 202, 407-415), where the two tandem tetO1 copies were separated by spacer nucleotide having the Seq ID No: 11 (5′-GGCGTTACTGGTCCTACG-3′), were cloned into the BamHI site of pEXAMPLE3.1 to yield pEXAMPLE3.2 (FIG. 21). To create pEXAMPLE3.3 (FIG. 22), an expression cassette containing the Tn10 tet repressor protein coding sequence having Seq ID No: 7 (EMBO 3, 539-43), fused to ERFP (pEGF-C1, Clontech) under the control of the GP64 promoter from AcMNPV (NCBI accession number NC_{—}001623) having Seq ID No: 8 was created by gene synthesis and subcloned into BstZ17I and KpnI digested pEXAMPLE3.2.

To create composite baculovirus bMON14272-pEXAMPLE3.3, composite bacmid DNA was isolated from DH10BAC following Tn7 transposition and transfected into SF21 insect cells according to (Nature Methods 3, 1021-1032). bMON14272-pEXAMPLE3.3 initial transfection virus and infected cells were then harvested and used for subsequent expression tests.

In FIG. 23A, the tetracycline concentrations required to induce polh-driven EGFP expression were investigated. Briefly, SF21 cells seeded at 70% confluency in a 6-well plate were infected with bMON14272-pEXAMPLE3.3 initial transfection virus at M.O.I 1.0 according to (Nature Methods 3, 1021-1032) in the presence of the indicated levels of tetracycline (Sigma T7660). Following 72 hours incubation, cells were lysed in 50 mM Tris, 50 mM NaCl, 0.1% NP40 pH 7.2, and absorbance was measured on a Beckman DU 640 spectrophotometer. As can be seen from FIG. 23A. tetracycline concentrations on the order of 0.1-1.0 μg/ml are sufficient to maximally induce expression of EGFP.

As a control, absorption of ERFP was also investigated (FIG. 238). Since, in this Example 4. no tetracycline regulatory elements are located in the vicinity of the tet-ERFP expression cassette, it was anticipated that no tetracycline dependent expression of ERFP would be observed. FIG. 23B indeed revealed no tetracycline dependent induction of ERFP expression.

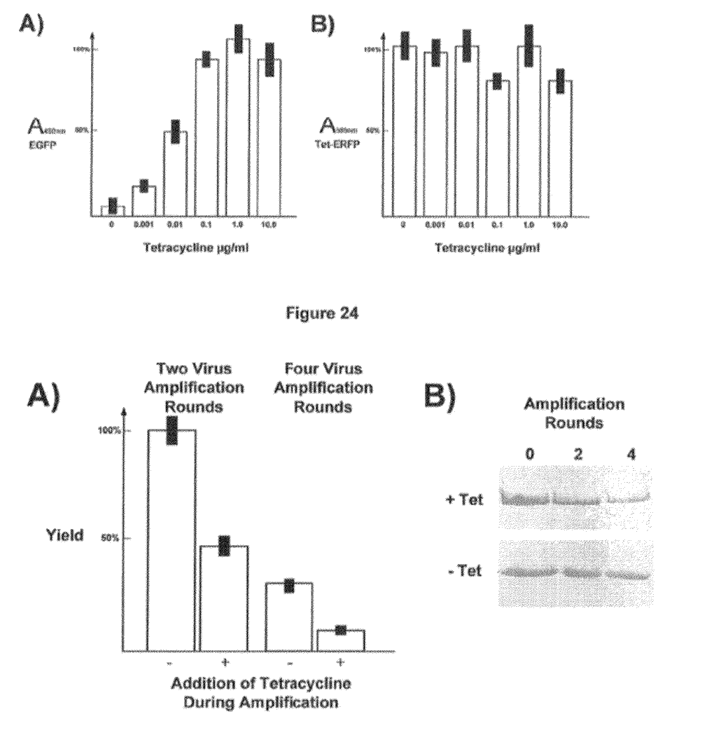

Expression tests to determine expression yield during scale up (FIG. 24) were carried out as follows. Initial transfection virus bMON14272-pEXAMPLE3.3 was serially passaged at multiplicity of infection (M.O.I) 0.1 for the indicated viral generations, either in the presence, or absence of 1.0 μg/ml tetracycline. In the absence of tetracycline, the tet-ERFP fusion protein (controllable transcription modulator protein) binds to its DNA binding site (transcriptional modulator response sequence) in C, repressing transcription—and hence expression—of EGFP (the recombinant protein under control of a polh promoter; a baculovirus very late promoter). If tetracycline is present during virus amplification it causes dissociation of the tet-ERFP fusion protein from its DNA binding site in C and consequently allows expression of EGFP. Accordingly, such conditions (virus amplification in the absence of tetracycline) act as a control to represent a prior-art baculovirus expression system that constitutively expresses recombinant protein during virus amplification.

Following each virus amplification round, virus was harvested at 48 hours post infection and used for both expression tests and for subsequent amplification rounds. For expression tests, SF21 cells seeded at 70% confluency in a 6-well plate were infected at M.O.I 1.0 according to (Nature Methods 3, 1021-1032) in the presence of 1.0 μg/ml tetracycline to induce EGFP. Following 72 hours incubation, cells were lysed in 50 mM Tris, 50 mM NaCl, 0.1% NP40 pH 7.2, and absorbance at 490 nm (for EGFP) was measured on a Beckman DU 640 spectrophotometer. Cells from two amplification rounds carried out in the absence of 1.0 μg/ml tetracycline during amplification (FIG. 24A, leftmost column) were diluted to reach absorption at 490 nm of 1.0 and a comparable number of cells from the other conditions outlined above and in the figure were compared for absorption at 490 nm.

As can be seen from FIG. 24A, relative to each control experiment where virus amplification was conducted in the presence of tetracycline (to mimic a prior-art baculovirus expression system that constitutively expresses recombinant protein), the yield of recombinant protein (EGFP) produced by the present invention is significantly greater. Indeed, after two rounds of amplification, the yield of recombinant protein from the inducible baculovirus expression system is over about 100 relative-percentage greater than that produced from the control that mimics a prior art system which constitutively expresses recombinant protein during virus amplification (100% vs 48%). After four rounds of virus amplification, this improvement is more pronounced, with about 115% greater relative percentage yield of recombinant protein for the system of the invention (32%) compared to the control system (15%). The amount of recombinant protein produced from an equivalent amount of cells of each system, as detected and visually quantified by western blotting (FIG. 24B) of samples tested in FIG. 24A using an anti-EGFP (Abcam) antibody, supports the results of the absorption analysis.

### Example 5

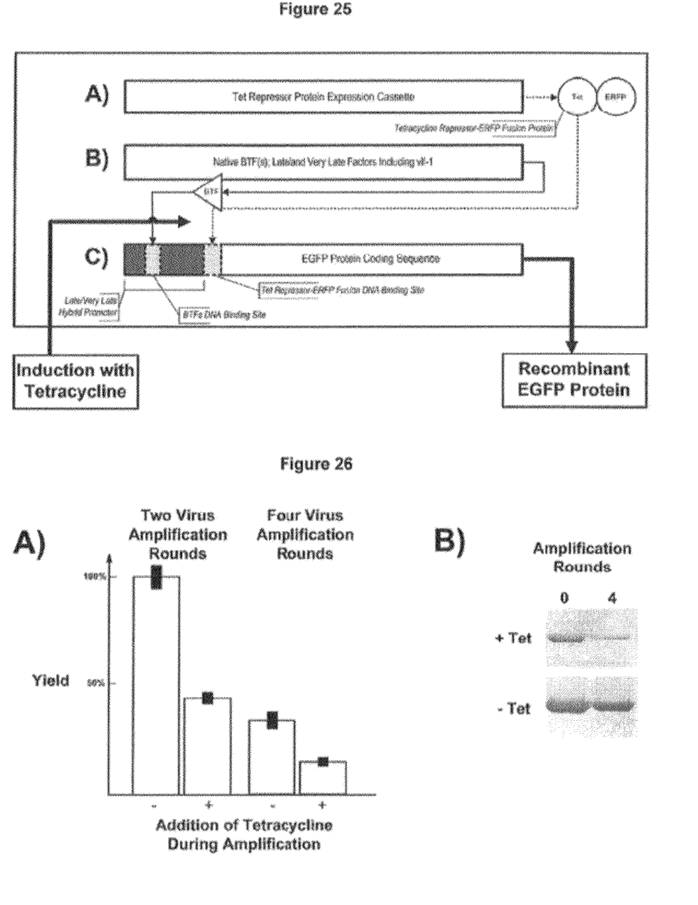

In Example 5 (FIG. 25) expression cassettes A, B, and C are identical to those used in Example 4, except that in Example 5, cassette C is driven by a late/very late hybrid promoter.

Baculovirus late promoter Pcap (Gene. 91 (1990) 87-94), having Seq ID No: 12, was created by gene synthesis to contain 5′ BgIII and 3′ BamHI restriction enzyme sites and was cloned into BamHI digested pFBDM from (Nature Biotechnology 22, 1583-1587). This created a late/very late hybrid promoter. Subsequent cloning steps were carried out exactly as described for generation of pEXAMPLE3.1, pEXAMPLE3.2, and pEXAMPLE3.3.

Composite baculovirus generation for this late/very late hybrid promoter-containing virus was also carried out in identical fashion as with Example 4 (FIG. 19), in that the cassette A and C containing transfer vector was fused with the cassette C containing bMON14272 in DH10BAC cells using Tn7 transposition.

Expression tests to determine expression stability of the late/very late hybrid promoter driven baculovirus during scale up (FIG. 26) were also carried out as with Example 4. Cells from two amplification rounds carried out in the absence of 1.0 μg/ml tetracycline during amplification (FIG. 26A, leftmost column) were diluted to reach absorption at 490 nm of 1.0 and a comparable number of cells from the other conditions outlined above were compared for absorption at 490 nm.

As can be seen from FIG. 26A, using a different baculorvirus promoter (in this case, a hybrid promoter containing sequences from both very late and late promoters from baculovirus), the relative increase in yield of recombinant protein for the inducible baculovirus expression system of the invention (absence of tetracycline) compared to the control to mimic a prior art system (presence of tetracycline) is similar to that seen in Example 4 (e.g., see FIG. 24A) after both two (100% vs 43%) and four (31% vs 21%) rounds of virus amplification. This Experiment 5 demonstrates that promoters comprising baculovirus late promoter may be used for the present invention. The amount of recombinant protein produced from an equivalent amount of cells of each system, as detected and visually quantified by western blotting (FIG. 26B) of samples tested in FIG. 26A using an anti-EGFP (Abcam) antibody, supports the results of the absorption analysis.

### Example 6

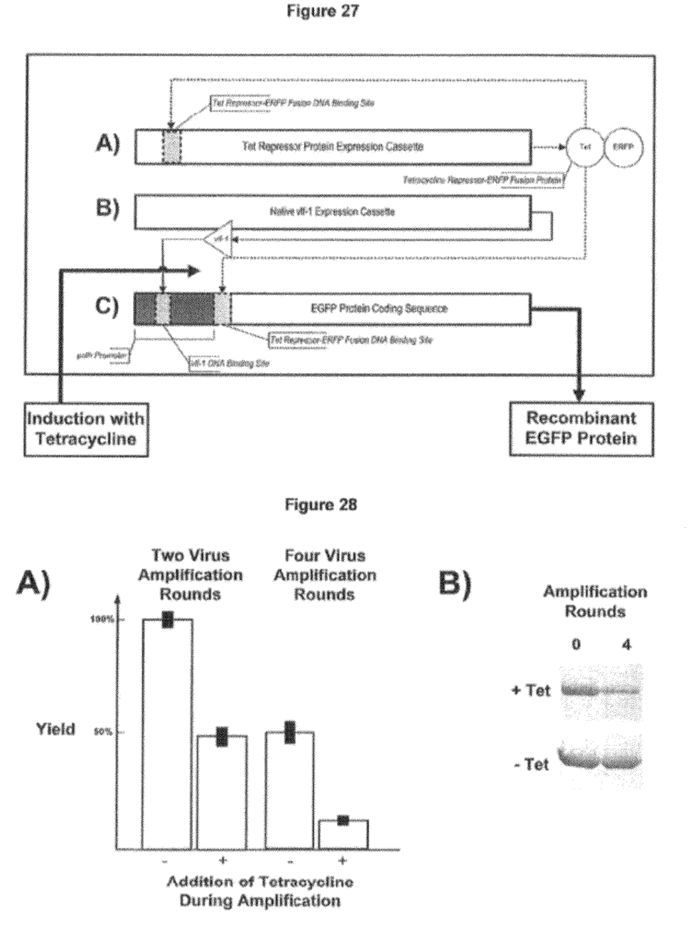

In Example 6 (FIG. 27) expression cassettes A, B, and C are identical to those used in Example 4, except that in Example 6, the tet-ERFP expression cassette is auto repressed through the presence of a tet repressor DNA binding site (a [second] transcriptional modulatory response element) in A.

To create a cassette A and C containing transfer vector for Example 6, cloning steps were carried out exactly as described in Example 4 for generation of pEXAMPLE3.1, pEXAMPLE3.2, and pEXAMPLE3.3 with one exception. Namely, for generation of pEXAMPLE3.3 the gene synthesis product encoding cassette A was engineered to contain a tet repressor DNA recognition sequence, as a (second) transcriptional modulatory response element, between the GP64 promoter and the tet-EGFP fusion protein. As in C, the tet repressor DNA recognition sequence in A comprises two tandem copies of the tetO1 sequence having Seq ID No: 6 (5′-ACTCTATCATTGATAGAGT-3′ from J. Mol. Biol. 202, 407-415), separated by spacer nucleotide sequence 5′-GGCGTTACTGGTCCTACG-3′ (Seq ID No: 11)

Composite baculovirus generation for this auto repressing cassette A containing DNA was also carried out in identical fashion as with Example 4 (FIG. 19), in that the cassette A and C containing transfer vector was fused with the cassette C containing bMON14272 in DH10BAC cells using Tn7 transposition.

Expression tests to determine expression stability of the late/very late hybrid promoter driven baculovirus during scale up (FIG. 28) were also carried out as with Example 4. Cells from two amplification rounds carried out in the absence of 1.0 μg/ml tetracycline during amplification (FIG. 28A, leftmost column) were diluted to reach absorption at 490 nm of 1.0 and a comparable number of cells from the other conditions outlined above were compared for absorption at 490 nm.

As can be seen from FIG. 28A, using an embodiment of the invention where the transcription of the transcriptional modulator protein is also controlled by the inclusion of a (second) transcriptional modulator response element in expression cassette A, the relative increase in yield of recombinant protein for such a system compared to a control (that mimics a prior art system which constitutively expresses recombinant protein during virus amplification) is similar to the previous experiments after two rounds of virus amplification (100% vs 47%) but is surprisingly enhanced after four rounds of virus amplification (49% vs 16%). This corresponds to about a 200 relative-percentage increase in protein production—after four rounds of virus amplification—for the system of the invention (49%) compared to a control that mimics a prior art system (16%), and indeed after four rounds of virus amplification the inventive system maintains a similar yield of protein production (49%) to that for the control (i.e. analogous to a prior art system) after only two rounds of amplification (47%). Such increases in yield of recombinant protein production after virus amplification, especially when conducted at industrial scale, are significant improvements in respect of production efficiency and reduction in the cost of goods. Indeed, the loss of efficiency of recombinant protein production for prior art baculovirus expression systems during virus amplification often means that they are not amplified, and particularly not by such numbers of rounds, during industrial production. The amount of recombinant protein produced from an equivalent amount of cells of each system, as detected and visually quantified by western blotting (FIG. 28B) of samples tested in FIG. 28A using an anti-EGFP (Abcam) antibody, confirmed the results of the absorption analysis.

Sequence Listing

Seq ID No: 1.
polyhedrin promoter
atcatggaga taattaaaat gataaccatc tcgcaaataa ataagtattt tactgttttc
gtaacagttt tgtaataaaa aaacctataa atattccgga ttattcatac cgtcccacca
tcgggcgcg
Seq ID No: 2.
P10 promoter
tatacggacc tttaattcac ccaacacaat atattatagt taaataagaa ttattatcaa
atcatttgta tattaattaa aatactatac tgtaaattac attttattta caatcactcg
acg
Seq ID No: 3.
gene vlf-1 (AcMNPV), translated amino acid sequence:
MHGFNVRNEN NFNSWKIKIQ SAPRFESVFD LATDRQRCTP DEVKNNSLWS KYMFPKPFAP
TTLKSYKSRF IKIVYCSVDD VHLEDMSYSL DKEFDSIENQ TLLIDPQELC RRMLELRSVI
KETLQLTINF YTNMMNLPEY KIPRMVMLPR DKELKNIREK EKNLMLKNVI DTILNFINDK
IKMLNSDYVH DRGLIRGAIV FCIMLGTGMR INEARQLSVD DLNVLIKRGK LHSDTINLKR
KRSRNNTLNN IKMKPLELAR EIYSRNPTIL QISKNTSTPP KDFRRLLEES GVEMERPRSN
MIRHYLSSNL YNSGVPLQKV AKLMNHESSA STKHYLNKYN IGLDETSSEE ENNNDDDDAQ
HNRNSSGSSG ESLLYYRNE
Seq ID No: 4.
gene vlf-1 (AcMNPV) DNA sequence (vlf-1 coding region and 3′ untranslated):
atgaacggttttaatgttcgcaacgaaaacaattttaattcttggaaaataaaaattcaatccgctccccggttc
gagtccgtgttcgatttggccaccgatcggcaacgatgcacgcccgacgaggtgaaaaacaacagtctgtggagc
aagtacatgttccccaaaccgtttgcgcccaccactttaaaaaggttacaagtctcgttcattaaaattgtgtac
tgctcggtagacgatgttcacctggaagacatgtcgtactcgttggacaaggagtttgactcgatagaaaaccaa
acacttctcattgatccccaagaactgtgcaggcgcatgctcgaacttcgctcggtcaccaaagaaacactacag
ttgactataaacttttacaccaacatgatgaacttgcccgaatacaaaattccccgcatggttatgctgccgcgc
gacaaggagctcaaaaatatcagggaaaaggaaaagaatttaatgcttaaaaacgtaatagataccatattaaat
tttattaatgataaaattaaaatgctcaacagcgattatgttcacgaccgcggtctaattaggggcgcgatagtg
ttttgcatcatgttagggacgggtatgcgaatcaacgaagcgcgccaactcagcgtggacgatctcaacgtgcta
attaaaagaggaaaactgcacagcgacacgattaatttaaagcgaaaacgcagtcgtaataacacactcaacaac
atcaaaatgaaaccgttggaattggcacgcgagatttattcacgaaacccgaccattttgcaaatatctaaaaac
acctcgacgcccttcaaagatttcaggcgactccttgaagagtcgggcgtcgagatggaacggccgcgcagcaac
atgataagacattatttgagcagtaacctatacaatagcggcgtgcctttacaaaaagtggccaaattaatgaac
cacgaatcctccgcaagcaccaaacattacttgaacaaatacaatataggtttagacgaaacgagcagcgaagag
gagaacaacaacgacgacgacgacgcgcagcataatcgcaattcgtccggttcgtcgggagaatcgttgttgtac
tatcgcaacgaatagagtaagggaataaaaatgaatttatatttgttgttgggcgcactggccatatttagccta
gtgtatgacaaaaaggaaaacagcatctttttgtatctgctcatattgtttctcgtgtttattatcgtcagcccg
gccattataagtaagaacaccgagtctaccgtagaagacataccgagtcataaagctaagagcgtccgaaaaaaa
tt
Seq ID No: 5.
vlf-1 promoter (AcMNPV)
ggcgctcaac gacgatactg ccagcgaacc gcaacaattt agcgagcccg ttcacaaaat
gccaataaac gacatggtgg gctatgacaa cacgacgagc aacgtgtcgg cgggaattat
aattttaatt agtgtagtcg cttttatagc tttattctta ctgctgtatg taatatatta
tttcgtaata ttaagagaac aacaacaata ttcggatagt attgacaccg attctccttt
tgtttttaat aaatttgatt aacaca
Seq ID No: 6.
tetO1 sequence:
actctatcat tgatagagt
Seq ID No: 7.
TetR protein aa sequence
MSRLDKSKVI NSALELLNEV GIEGLTTRKL AQKLGVEQPT LYWHVKNKRA LLDALAIEML
DRHHTHFCPL EGESWQDFLR NNAKSFRCAL LSHRDGAKVH LGTRPTEKQY ETLENQLAFL
CQQGFSLENA LYALSAVGHF TLGCVLEDQE EQVAKEERET PTTDSMPPLL RQAIELFDHQ
GAEPAFLFGL ELIICGLEKQ LKCESGS
Seq ID No: 8.
GP64 promoter DNA sequence (AcMNPV):
cgactgagcg tccgtgttca tgatcccgtt tttataacag ccagataaaa ataatcttat
caattaagat aaaaagataa gattattaat ctaacaacgt gccttgtgtc acgtaggcca
gataacggtc gggtatataa gatgcctcaa tgctactagt aaatcagcca caccaaggct
tcaataagga acacacaagc aag
Seq ID No: 9.
vlf-1 weak promoter based on vhspRVvlf1 promoter DNA sequence
ggcgctcaac gacgatactg ccagcgaacc gcaacaattt agcgagcccg ttcacaaaat
gccaataaac gacatggtgg gctatgacaa cacgacgagc aacgtgtcgg cgggaattat
aattttaatt agtgtagtcg cttttatagc tttattctta ctgctgtatg taatatatta
tttcgtaata ttaagagaac aacaacaata ttcggatagt attgacaccg attctccttt
tgtttttaat aaatttgatt aacctgtgtg tgagttcttc ctcggtaacg acttgttgaa
agtattcaga gttctcttct tgtcttcaat aatgacttct tggttgattt cagtagttgc
agtttttagt ttaattactt ggttgttggt tacttttaat tgattcactt taacttgcac
tttattgcag attgtttagc ttgttcgctg cgcttgtttg ttgctcagct tacgcttcgc
gatgtgttca ctttgcttgt ttgaattgaa ttgacgctcc gtcgacgaag cgcctctatt
tatactccgg cgctcttttc gcgaacattc gcaggccgcg ctctctcgaa gcaacgagaa
tagcgtgccg tttactgtgc gacagagtga gagagcaata gtacagagag ggagagtcac
aaaacgaata gagaataacg gccagagaaa tttctcgagt tttctttctg gcaaacaaat
gcctcgcgca acaaccaggt ttgttttggg ggttctagaa tattctttat ttatatatat
actttatttt ggaaatttct ttataaatac ggctgcttaa gttaattatg ttagacataa
tcgaagggtt tgttagcgga tgttgtccgc cagaaaggcc tatggaactt tgacaagata
ttcttcaaaa tgtatttaca tactaactta aaaaagctat ttatttatta gattaataca
gacaattgca tgcagatgat tgttagtgtt tttaatttaa aattacgtac aggttgtcaa
gactgttgtt gtaca
Seq ID No: 10.
vlf-1 coding sequence codon optimized for insect cell expression
atgaacggtt tcaacgtgcg taacgagaac aacttcaact cctggaagat caagatccag
tccgctcccc gtttcgagtc cgtgttcgac ctggctaccg accgtcagcg ttgcaccccc
gacgaggtga agaacaactc cctgtggtcc aagtacatgt tccccaagcc cttcgctccc
accaccctga agtcctacaa gtcccgtttc atcaagatcg tgtactgctc cgtggacgac
gtgcacctgg aggacatgtc ctactccctg gacaaggagt tcgactccat cgagaaccag
accctgctga tcgaccccca ggagctgtgc cgtcgtatgc tggagctgcg ttccgtgacc
aaggagaccc tgcagctgac catcaacttc tacaccaaca tgatgaacct gcccgagtac
aagatccccc gtatggtgat gctgccccgt gacaaggagc tgaagaacat ccgtgagaag
gagaagaacc tgatgctgaa gaacgtgatc gacaccatcc tgaacttcat caacgacaag
atcaagatgc tgaactccga ctacgtgcac gaccgtggtc tgatccgtgg tgctatcgtg
ttctgcatca tgctgggtac cggtatgcgt atcaacgagg ctcgtcagct gtccgtggac
gacctgaacg tgctgatcaa gcgtggtaag ctgcactccg acaccatcaa cctgaagcgt
aagcgttccc gtaacaacac cctgaacaac atcaagatga agcccctgga gctggctcgt
gagctctact cccgtaaccc caccatcctg cagatctcca agaacacctc cacccccttc
aaggacttcc gtcgtctgct ggaggagtcc ggtgtggaga tggagcgtcc ccgttccaac
atgatccgtc actacctgtc ctccaacctg tacaactccg gtgtgcccct gcagaaggtg
gctaagctga tgaaccacga gtcctccgct tccaccaagc actacctgaa caagtacaac
atcggtctgg acgagacctc ctccgaggag gagaacaaca acgacgacga cgacgctcag
cacaaccgta actcctccgg ttcctccggt gagtccctgc tgtactaccg taacgagtaa
Seq ID No: 11.
spacer nucleotide sequence
GGCGTTACTGGTCCTACG
Seq ID No: 12.
late baculovirus promoter sequence for creation of hybrid late/very late promoter:
TTATGTTATTGCAAGCGCTCTGAATAGGTATACGAGTGCGAAAGCCGTTTTCGTCGTACAAATCGA
AATATTGTTGTGCCAGCGAATAATTAGGAACAATATAAGAATTTAAAATTTTATACAACAAATCTTG
GCTAAAATTTATTGAATAAGAGATTTCTTTCTCAATCACAAAATCGCCGTAGTCCATATTTATAACG
GCAACAC

1. An inducible baculovirus expression system, wherein recombinant protein expression can be repressed during virus amplification in insect cells, comprising:
a) at least one expression cassette A containing a promoter and an open reading frame coding for a controllable transcriptional modulator protein,
b) at least one expression cassette B containing a promoter and an open reading frame coding for a factor which regulates transcriptional activity of a baculovirus late and/or very late promoter,
c) at least one expression cassette C containing an open reading frame coding for a recombinant protein under the control of a baculovirus late and/or very late promoter, and
d) a transcriptional modulator response element;
wherein said controllable transcriptional modulator protein reversibly interacts with said transcriptional modulator response element in one condition, and differently in a second condition, thereby modulating the transcription of expression cassette B or expression cassette C.

2. The inducible baculovirus expression system of claim 1, further characterised by one or more of:
said baculovirus late and/or very late promoter is a baculovirus very late promoter and said factor is a factor which regulates transcriptional activity of a baculovirus very late promoter (“VLTF”);
wherein, with respect to expression cassette B, transcriptional activity of said baculovirus late and/or very late promoter decreases with lower than wild type levels of said factor in insect cells; and/or
wherein in the repressed (off) state, expression levels of said recombinant protein cassette C is lower compared to a non-inducible baculovirus expression system with an expression cassette B containing an open reading frame coding for said factor under the control of its original promoter, and an expression cassette C as defined above, but without an expression cassette A and/or without its corresponding transcriptional modulator response element.

3. The inducible baculovirus expression system of claim 1, wherein the transcription modulator response element is in expression cassette C, or in expression cassette B.

4. (canceled)

5. The inducible baculovirus expression system of claim 1, comprising a further expression cassette B′ containing an open reading frame coding for said factor under the control of a weak promoter producing lower than wild type levels of said factor allowing baculovirus replication, or a further expression cassette B′ containing a promoter and an open reading frame coding for a modified factor leading to lower than wild type transcriptional activity of a baculovirus late and/or very late promoter, and wherein said modified factor allows baculovirus replication.

6. (canceled)

7. The inducible baculovirus expression system of claim 5, wherein said further expression cassette B′ is not inducible.

8. The inducible baculovirus expression system of claim 1, wherein the transcriptional modulator protein is a transcriptional repressor protein and further wherein said modulator response element is a transcriptional repressor response element.

9. (canceled)

10. The inducible baculovirus expression system of claim 3, wherein the transcriptional modulator protein is a transcriptional activator protein and expression cassette B contains an open reading frame coding for said factor under the control of a weak promoter producing lower than wild type levels of said factor, and further wherein said modulator response element is a transcription activator response element capable of activating transcription of expression cassette B.

11. (canceled)

12. The inducible baculovirus expression system of claim 1, wherein said factor encoded by expression cassette B is vlf-1 of Seq ID No: 3 or a transcriptionally functional homolog thereof or wherein said modified factor expressed by expression cassette B′ is a modified vlf-1 protein.

13. (canceled)

14. (canceled)

15. (canceled)

16. The inducible baculovirus expression system of claim 5, wherein said weak promoter of expression cassettes B or B′ has a sequence of Seq ID No: 9.

17. (canceled)

18. (canceled)

19. (canceled)

20. The inducible baculovirus expression system of claim 1, wherein any one of said expression cassettes is contained in a transfer vector suitable for fusion with modified baculovirus DNA, in a modified baculovirus DNA, in a genomic baculovirus DNA, in a separate chromosomal DNA within cells infected with the baculovirus, or in a non-chromosomal DNA within a cell infected with a baculovirus.

21. (canceled)

22. (canceled)

23. (canceled)

24. (canceled)

25. (canceled)

26. (canceled)

27. An insect cell comprising the inducible baculovirus expression system of claim 1.

28. A method of producing a recombinant protein in insect cells comprising;
a) introducing the inducible baculovirus expression system of claim 1 into insect cells,
b) culturing said insect cell under conditions where recombinant protein expression is repressed,
c) inducing protein production by activating an activator or deactivating a repressor;
d) harvesting said recombinant protein.

29. The method of claim 28, wherein recombinant protein expression is repressed by a transcriptional repressor protein binding to a transcriptional repressor response element in
a) expression cassette C encoding the recombinant protein, or
b) expression cassette B encoding a late and/or very late transcription factor which regulates transcriptional activity of a baculovirus late and/or very late promoter.

30. The method of claim 28 wherein recombinant protein expression is repressed by lack of transcriptional activator protein interaction with a transcriptional activator response element, wherein said interaction activates transcription of expression cassette B containing the open reading frame encoding said factor under the control of a weak promoter.

31. The insect cell of claim 27, wherein the insect cell is selected from the group consisting of insect cells derived from Spodoptera frugiperda, Trichoplusia ni, Plutella xylostella, Manduca sexta, and Mamestra brassicae.

32. (canceled)

33. A kit for an inducible baculovirus expression system in insect cells comprising:
a) a transfer vector comprising at least one expression cassette C(-) containing a baculovirus late and/or very late promoter, wherein said expression cassette is intended for expressing a recombinant protein under the control of said promoter,
b) a modified baculovirus DNA comprising an expression cassette B containing a promoter and an open reading frame encoding a factor which regulates transcriptional activity of a baculovirus late and/or very late promoter,
wherein transcriptional activity of said baculovirus late and/or very late promoter decreases with lower than wild type levels of said factor in insect cells; and
c) an expression cassette A encoding a transcriptional repressor protein either on the transfer vector of component a) or on the modified baculovirus DNA of component b);
wherein either said expression cassette C(-) or B further comprises a transcriptional repressor response element or comprising:
a) a transfer vector comprising at least one expression cassette C(-) into which an open reading frame encoding a recombinant protein can be cloned and wherein the expression of said recombinant protein is under the control of a baculovirus late and/or very late promoter,
b) a modified baculovirus DNA comprising an expression cassette B containing a transcriptional activator response element and an open reading frame encoding a late and/or very late transcription factor which regulates transcriptional activity of a baculovirus late and/or very late promoter under the control of a weak promoter,
wherein the weak promoter produces lower than wild type levels of said factor allowing viral replication, but causing decreased transcriptional activity of said baculovirus late and/or very late promoter in expression cassette C in insect cells;
c) an expression cassette A encoding a transcriptional activator protein either on the transfer vector of component a) or on the modified baculovirus DNA of component b).

34. (canceled)

35. The kit of claim 33, wherein said modified baculovirus DNA further comprises an expression cassette B′ containing
a) an open reading frame coding for said factor under the control of a weak promoter producing lower than wild type levels of said factor allowing baculovirus replication; or
b) a promoter and an open reading frame coding for a modified factor leading to lower than wild type transcriptional activity of a baculovirus late and/or very late promoter, and wherein said modified factor allows baculovirus replication.

36. The kit of claim 33, wherein expression cassette C(-) further contains an open reading frame for expressing said recombinant protein under the control of said promoter.

37. (canceled)

38. (canceled)

39. A recombinant nucleic acid comprising at least: (i) the promoter of expression cassette C of the inducible baculovirus expression system of claim 1; (ii) a transcriptional modulator response element; and (iii) a cloning site for a nucleic acid, wherein said transcriptional modulator response element:
is not a hormone receptor response element;
binds to a bacterial controllable transcriptional modulator protein;
is, or is derived from, a bacterial transcriptional modulator response element; and/or
is a transcriptional repressor response element.

40. (canceled)

41. (canceled)

42. A Modified baculovirus DNA comprising expression cassette B of the inducible baculovirus expression system of claim 1, wherein:
a) the expression cassette B contains an open reading frame coding for said factor under the control of a weak promoter and further comprises a transcriptional activator response element, or
b) the expression cassette B comprises a transcriptional repressor response element.

43. The recombinant nucleic acid of claim 39, further comprising an expression cassette A of the inducible baculovirus expression system of claim 1.

44. The method of claim 28, wherein the culturing step b) comprises conditions under which baculovirus is amplified in said insect cells, under which the number of insect cells increases and/or under which the number of baculovirus particles is increased.

45. (canceled)

46. The method of claim 44, wherein said culture conditions are maintained until the number of insect cells is between about 10⁸ and 10¹³ and/or until the number of baculovirus particles is between 10⁸-10¹³.

47. The method of claim 44, wherein said conditions are maintained until the total volume of culture is between 0.1 L and 10,000 L, and/or are maintained for a period of time that is between 1 day and 3 weeks after introduction step of a).

48. The method of claim 28, wherein the induction step of c) comprises the presence of an inducer molecule with said inducible baculovirus expression system, preferably by addition of said inducer molecule to the culture conditions of said insect cells.

49. (canceled)

50. A method of repressing transcription of an open reading frame coding for a recombinant protein during baculovirus amplification in insect cells, said method comprising:
a. Providing insect cells of claim 27, and
b. Maintaining said insect cells under conditions wherein the transcription of the open reading frame contained in expression cassette C of said inducible baculovirus expression system is repressed by the controllable transcriptional modulator protein encoded by expression cassette A of said inducible baculovirus expression system;
and, optionally, wherein said conditions allow replication of baculovirus particles in said insect cells.

51. (canceled)

52. (canceled)

53. (canceled)

54. (canceled)

55. (canceled)

56. (canceled)

57. The method of claim 50, wherein said repression is brought about by the controllable transcriptional modulator protein (encoded by expression cassette A of said inducible baculovirus expression system) reversibly interacting with the transcriptional modulator response element (of said inducible baculovirus expression system) in one condition, and differently in a second condition, thereby modulating the transcription of expression cassette B or expression cassette C of said inducible baculovirus expression system.

58. The method of claim 57, wherein interaction of said controllable transcriptional modulator protein to said transcriptional modulator response element causes said repression, and optionally wherein said transcriptional modulator response element is in expression cassette C of said inducible baculovirus expression system.

59. The method of claim 50 further comprising the step of inducing transcription of the open reading frame in expression cassette C that encodes a recombinant protein.

60. (canceled)

61. (canceled)

62. (canceled)

63. (canceled)

64. A kit comprising:
the recombinant nucleic acid of claim 39; and separately
at least one other component for the construction and/or use of an inducible baculovirus expression system.

65. The kit of claim 64, wherein said at least one other component comprises:
the expression cassette A of the inducible baculovirus expression system of claim 1;
an insect cell from the group consisting of insect cells derived from Spodoptera frugiperda, Trichoplusia ni, Plutella xylostella, Manduca sexta, and Mamestra brassicae;
an inducer molecule that modulates the reversible interaction of a controllable transcriptional modulator protein with a transcriptional modulator response element; and
instructions describing how to construct and/or use the inducible baculovirus expression system of claim 1.


SEQUENCE LISTING (12 sequences)

SEQ ID NO: 1 - DNA, 129 bp, Autographa californica multicapsid nuclear polyhedrosis virus
atcatggagataattaaaatgataaccatctcgcaaataaataagtattttactgttttcgtaacagttttgtaataaaaaaacctataaatattccggattattcataccgtcccaccatcgggcgcg

SEQ ID NO: 2 - DNA, 123 bp, Autographa californica multicapsid nuclear polyhedrosis virus
tatacggacctttaattcacccaacacaatatattatagttaaataagaattattatcaaatcatttgtatattaattaaaatactatactgtaaattacattttatttacaatcactcgacg

SEQ ID NO: 3 - protein, 379 aa, Autographa californica multicapsid nuclear polyhedrosis virus
MNGFNVRNENNFNSWKIKIQSAPRFESVFDLATDRQRCTPDEVKNNSLWSKYMFPKPFAPTTLKSYKSRFIKIVYCSVDDVHLEDMSYSLDKEFDSIENQTLLIDPQELCRRMLELRSVTKETLQLTINFYTNMMNLPEYKIPRMVMLPRDKELKNIREKEKNLMLKNVIDTILNFINDKIKMLNSDYVHDRGLIRGAIVFCIMLGTGMRINEARQLSVDDLNVLIKRGKLHSDTINLKRKRSRNNTLNNIKMKPLELAREIYSRNPTILQISKNTSTPFKDFRRLLEESGVEMERPRSNMIRHYLSSNLYNSGVPLQKVAKLMNHESSASTKHYLNKYNIGLDETSSEEENNNDDDDAQHNRNSSGSSGESLLYYRNE

SEQ ID NO: 4 - DNA, 1352 bp, Autographa californica multicapsid nuclear polyhedrosis virus
atgaacggttttaatgttcgcaacgaaaacaattttaattcttggaaaataaaaattcaatccgctccccggttcgagtccgtgttcgatttggccaccgatcggcaacgatgcacgcccgacgaggtgaaaaacaacagtctgtggagcaagtacatgttccccaaaccgtttgcgcccaccactttaaaaagttacaagtctcgattcattaaaattgtgtactgctcggtagacgatgttcacctggaagacatgtcgtactcgttggacaaggagtttgactcgatagaaaaccaaacacttctcattgatccccaagaactgtgcaggcgcatgctcgaacttcgctcggtcaccaaagaaacactacagttgactataaacttttacaccaacatgatgaacttgcccgaatacaaaattccccgcatggttatgctgccgcgcgacaaggagctcaaaaatatcagggaaaaggaaaagaatttaatgcttaaaaacgtaatagataccatattaaattttattaatgataaaattaaaatgctcaacagcgattatgttcacgaccgcggtctaattaggggcgcgatagtgttttgcatcatgttagggacgggtatgcgaatcaacgaagcgcgccaactcagcgtggacgatctcaacgtgctaattaaaagaggaaaactgcacagcgacacgattaatttaaagcgaaaacgcagtcgtaataacacactcaacaacatcaaaatgaaaccgttggaattggcacgcgagatttattcacgaaacccgaccattttgcaaatatctaaaaacacctcgacgcccttcaaagatttcaggcgactccttgaagagtcgggcgtcgagatggaacggccgcgcagcaacatgataagacattatttgagcagtaacctatacaatagcggcgtgcctttacaaaaagtggccaaattaatgaaccacgaatcctccgcaagcaccaaacattacttgaacaaatacaatataggtttagacgaaacgagcagcgaagaggagaacaacaacgacgacgacgacgcgcagcataatcgcaattcgtccggttcgtcgggagaatcgttgttgtactatcgcaacgaatagagtaagggaataaaaatgaatttatatttgttgttgggcgcactggccatatttagcctagtgtatgacaaaaaggaaaacagcatctttttgtatctgctcatattgtttctcgtgtttattatcgtcagcccggccattataagtaagaacaccgagtctaccgtagaagacataccgagtcataaagctaagagcgtccgaaaaaaatt

SEQ ID NO: 5 - DNA, 267 bp, Autographa californica multicapsid nuclear polyhedrosis virus
ggcgctcaacgacgatactgccagcgaaccgcaacaatttagcgagcccgttcacaaaatgccaataaacgacatggtgggctatgacaacacgacgagcaacgtgtcggcgggaattataattttaattagtgtagtcgcttttatagctttattcttactgctgtatgtaatatattatttcgtaatattaagagaacaacaacaatattcggatagtattgacaccgattctccttttgtttttaataaatttgattaaccaca

SEQ ID NO: 6 - DNA, 19 bp, E. coli
actctatcattgatagagt

SEQ ID NO: 7 - protein, 207 aa, E. coli
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 8 - DNA, 203 bp, Autographa californica multicapsid nuclear polyhedrosis virus
cgactgagcgtccgtgttcatgatcccgtttttataacagccagataaaaataatcttatcaattaagataaaaagataagattattaatctaacaacgtgccttgtgtcacgtaggccagataacggtcgggtatataagatgcctcaatgctactagtaaatcagtcacaccaaggcttcaataaggaacacacaagcaag

SEQ ID NO: 9 - DNA, 1035 bp, Artificial sequence
  synthetic
ggcgctcaacgacgatactgccagcgaaccgcaacaatttagcgagcccgttcacaaaatgccaataaacgacatggtgggctatgacaacacgacgagcaacgtgtcggcgggaattataattttaattagtgtagtcgcttttatagctttattcttactgctgtatgtaatatattatttcgtaatattaagagaacaacaacaatattcggatagtattgacaccgattctccttttgtttttaataaatttgattaacctgtgtgtgagttcttcctcggtaacgacttgttgaaagtattcagagttctcttcttgtcttcaataatgacttcttggttgatttcagtagttgcagtttttagtttaattacttggttgttggttacttttaattgattcactttaacttgcactttattgcagattgtttagcttgttcgctgcgcttgtttgttgctcagcttacgcttcgcgatgtgttcactttgcttgtttgaattgaattgacgctccgtcgacgaagcgcctctatttatactccggcgctcttttcgcgaacattcgcaggccgcgctctctcgaagcaacgagaatagcgtgccgtttactgtgcgacagagtgagagagcaatagtacagagagggagagtcacaaaacgaatagagaataacggccagagaaatttctcgagttttctttctggcaaacaaatgcctcgcgcaacaaccaggtttgttttgggggttctagaatattctttatttatatatatactttattttggaaatttctttataaatacggctgcttaagttaattatgttagagataatcgaagggtttgttagcggatgttgtccgccagaaaggcctatggaactttgacaagatattcttcaaaatgtatttacatactaacttaaaaaagctatttatttattagattaatacagacaattgcatgcagatgattgttagtgtttttaatttaaaattacgtacaggttgtcaagactgttgttgtaca

SEQ ID NO: 10 - DNA, 1140 bp, Artificial sequence
  synthetic
atgaacggtttcaacgtgcgtaacgagaacaacttcaactcctggaagatcaagatccagtccgctccccgtttcgagtccgtgttcgacctggctaccgaccgtcagcgttgcacccccgacgaggtgaagaacaactccctgtggtccaagtacatgttccccaagcccttcgctcccaccaccctgaagtcctacaagtcccgtttcatcaagatcgtgtactgctccgtggacgacgtgcacctggaggacatgtcctactccctggacaaggagttcgactccatcgagaaccagaccctgctgatcgacccccaggagctgtgccgtcgtatgctggagctgcgttccgtgaccaaggagaccctgcagctgaccatcaacttctacaccaacatgatgaacctgcccgagtacaagatcccccgtatggtgatgctgccccgtgacaaggagctgaagaacatccgtgagaaggagaagaacctgatgctgaagaacgtgatcgacaccatcctgaacttcatcaacgacaagatcaagatgctgaactccgactacgtgcacgaccgtggtctgatccgtggtgctatcgtgttctgcatcatgctgggtaccggtatgcgtatcaacgaggctcgtcagctgtccgtggacgacctgaacgtgctgatcaagcgtggtaagctgcactccgacaccatcaacctgaagcgtaagcgttcccgtaacaacaccctgaacaacatcaagatgaagcccctggagctggctcgtgagatctactcccgtaaccccaccatcctgcagatctccaagaacacctccacccccttcaaggacttccgtcgtctgctggaggagtccggtgtggagatggagcgtccccgttccaacatgatccgtcactacctgtcctccaacctgtacaactccggtgtgcccctgcagaaggtggctaagctgatgaaccacgagtcctccgcttccaccaagcactacctgaacaagtacaacatcggtctggacgagacctcctccgaggaggagaacaacaacgacgacgacgacgctcagcacaaccgtaactcctccggttcctccggtgagtccctgctgtactaccgtaacgagtaa

SEQ ID NO: 11 - DNA, 18 bp, Artificial sequence
  synthetic
ggcgttactggtcctacg

SEQ ID NO: 12 - DNA, 207 bp, Artificial sequence
  synthetic
ttatgttattgcaagcgctctgaataggtatacgagtgcgaaagccgttttcgtcgtacaaatcgaaatattgttgtgccagcgaataattaggaacaatataagaatttaaaattttatacaacaaatcttggctaaaatttattgaataagagatttctttctcaatcacaaaatcgccgtagtccatatttataacggcaacac